(19) United States
(12) Patent Application Publication
(10) Pub. No.: US 2010/0028358 A1
(43) Pub. Date: Feb. 4, 2010

(54) Compositions and Methods for Controlling Tissue Factor Signaling Specificity

(76) Inventors: Wolfram Ruf, San Diego, CA (US); Jassimuddin Ahamed, New York, NY (US); Henrik Versteeg, Leiden (NL); Barbara M. Mueller, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 12/084,225

(22) PCT Filed: Nov. 6, 2006
(86) PCT No.: PCT/US06/43313

§ 371 Date: Apr. 21, 2009

Related U.S. Application Data
(60) Provisional application No. 60/734,149, filed on Nov. 7, 2005.

(51) Int. Cl. A61K 39/395; G01N 33/53

(52) U.S. Cl. 424/141.1; 435/7.1

(57) ABSTRACT

Compositions and methods for treating a disease dependent upon tissue factor/factor VIIa signaling in a mammalian subject is provided. The methods comprise administering an inhibitor of tissue factor signaling to the mammalian subject. The inhibitor is effective in reducing the incidence of disease in the mammalian subject. Also provided are methods of screening for modulators of tissue factor/factor VIIa signaling.

### CROSS-REFERENCE TO RELATED APPLICATIONS

The subject patent application claims the benefit of priority to U.S. Provisional Patent Application No. 60/734,149 (filed Nov. 7, 2005). The full disclosures of the priority application are incorporated herein by reference in their entirety and for all purposes.

### STATEMENT OF GOVERNMENT SUPPORT

This invention was made by government support by Grant Nos. HL60472, HL31950, and HL16411 from the National Heart, Lung, and Blood Institute. The Government has certain rights in this invention.

### FIELD OF THE INVENTION

The invention generally relates to compositions and methods for treating a disease dependent upon tissue factor/factor VIIa (TF/VIIa) signaling in a mammalian subject. The methods comprise administering an inhibitor of tissue factor signaling to the mammalian subject. The inhibitor is effective in reducing or eliminating the incidence of disease or preventing its occurrence or recurrence, without interfering with hemostasis in the mammalian subject.

### BACKGROUND

The enzymatic complex of the cell surface receptor tissue factor (TF) with the serine protease factor VIIa activates physiological haemostasis and coagulation and concomitantly triggers protease activated receptor signaling in inflammation, tumour progression and angiogenesis. Mackman, Arterioscler. Thromb. Vasc. Biol. 24: 1015-1022, 2004; Riewald and Ruf, Crit. Care 7: 123-129, 2003; Belting et al., Arterioscler. Thromb. Vasc. Biol. 25: 1545-1550, 2005). How direct upstream TF-VIIa signaling determines pathology without being overridden by abundantly generated downstream coagulation proteases remained a fundamental, unresolved question in vascular biology. Kawabata et al., Br J Pharmacol., 144: 212-219, 2005; Namkung, et al., Gastroenterology, 126: 1844-1859, 2004; Fiorucci, et al., Proc Natl Acad Sci USA, 98: 13936-13941, 2001; Cocks et al., Nature, 398: 156-160, 1999.

TF binds and allosterically activates factor VIIa and assists in the assembly of the ternary TF-VIIa-X coagulation initiation complex that releases product Xa to generate thrombin. Norledge et al., Proteins 53: 640-648, 2003. The TF-VIIa complex also directly cleaves protease-activated receptor (PAR) 2. Camerer et al., Proc. Natl. Acad. Sci. USA 97: 5255-5260, 2000; Riewald and Ruf, Proc. Natl. Acad. Sci. USA 98: 7742-7747, 2001. Despite well established roles of thrombin signaling through PARs in haemostasis and inflammation, the activation of PARs by alternative proteases in vivo remains poorly defined. Coughlin, J. Thromb. Haemost. 3: 1800-1814, 2005. TF and PAR2 are closely linked, because TF cytoplasmic domain phosphorylation is downstream of PAR2 signaling and TF cytoplasmic domain-deleted mice show enhanced PAR2-dependent angiogenesis. Ahamed and Ruf, J. Biol. Chem. 279: 23038-23044, 2004; Belting et al., Nature Med. 10: 502-509, 2004. However, it is incompletely understood how TF-VIIa signaling is regulated and plays physiological roles independent of signaling by other downstream coagulation proteases. There are currently no angiogenesis inhibitors that are FDA-approved for treatment of angiogenesis-related disease. A need exists in the art to regulate or inhibit TF signaling related to angiogenesis, tumor cell growth, tumor metastasis or inflammation, while allowing blood hemostasis pathways to proceed normally.

### SUMMARY

The present invention generally relates to compositions and methods for treating disease in a mammalian subject, for example, angiogenesis-related disease, inflammation or neoplastic disease. The composition is an inhibitor of tissue factor signaling which does not interfere with hemostasis in the mammalian subject. The methods comprise administering an inhibitor of tissue factor signaling to the mammalian subject. The inhibitor is effective in reducing the incidence of an angiogenesis-related disease state, inflammation or neoplastic disease without increasing the risk of reduced coagulation or increased bleeding in the mammalian subject.

A method for treating a disease dependent upon tissue factor/factor VIIa signaling in a mammalian subject is provided comprising administering an inhibitor of tissue factor signaling to the mammalian subject in an amount effective to reduce or eliminate the disease or to prevent its occurrence or recurrence in the mammalian subject, wherein the inhibitor does not interfere with hemostasis in the mammalian subject. The diseases include, but are not limited to, angiogenesis-related disease, neoplastic disease, or inflammation. Neoplastic diseases or inflammation are included within the angiogenesis-related diseases. In one aspect, the inhibitor is an antibody or small chemical entity. In a detailed aspect, the inhibitor is monoclonal antibody 10H10 (MAb 10H10) produced by the hybridoma with ATCC access number HB9383.

A method for identifying a compound which modulates tissue factor signaling in cells is provided comprising the steps of contacting a test compound with a cell-based assay system comprising a cell expressing tissue factor capable of signaling, wherein tissue factor-dependent signaling is regulated by protein disulfide isomerase, providing factor VIIa to said assay system in an amount selected to be effective to activate tissue factor-dependent signaling, and detecting an effect of said test compound on tissue factor-dependent signaling in said assay system, effectiveness of said test compound in said assay being indicative of said modulation. In one aspect, method further comprises detecting an inhibitory effect of the test compound on tissue factor signaling. In a further aspect, the method further comprises detecting no effect of the test compound on tissue factor-mediated hemostasis. The cells include, but are not limited to, keratinocytes, melanoma, or endothelial cells. In a further aspect, the cell-based assay system signals responsiveness via protease activated receptor 2. In a further aspect, the test compound is an antibody or small chemical entity. In a detailed aspect, the compound inhibits the binding of MAb 10H10 to tissue factor. In a further detailed aspect, the compound does not inhibit the binding of monoclonal antibody 5G9 (MAb 5G9) produced by the hybridoma with ATCC access number HB9382 to tissue factor.

A method for treating angiogenesis in a mammalian subject is provided comprising administering a therapeutically effective amount of a compound which modulates signaling in cells via tissue factor-factor VIIa pathway, wherein said compound is an antagonist of tissue factor-factor VIIa signaling in a cell-based assay system, and said compound is effective to reduce or eliminate angiogenesis or to prevent its occurrence or recurrence in the mammalian subject. In a further aspect, the compound does not interfere with hemostasis in the mammalian subject. In one aspect, tissue factor-factor VIIa signaling is dependent on protein disulfide isomerase. In another aspect, tissue factor-factor VIIa signaling occurs via protease activated receptor 2. In a further aspect, the test compound is an antibody or small chemical entity. In a detailed aspect, the compound inhibits the binding of MAb 10H10 to tissue factor. In a further detailed aspect, the compound does not inhibit the binding of MAb 5G9 to tissue factor.

A method for treating a neoplastic disease in a mammalian subject is provided comprising, administering a therapeutically effective amount of a compound which modulates signaling in cells via tissue factor-factor VIIa pathway, wherein said compound acts as an antagonist of tissue factor-factor VIIa signaling in a cell-based assay system, and said compound is effective to reduce or eliminate the neoplastic disease or to prevent its occurrence or recurrence in the mammalian subject. In a further aspect, the compound does not interfere with hemostasis in the mammalian subject. In one aspect, tissue factor-factor VIIa signaling is dependent protein disulfide isomerase. In another aspect, tissue factor-factor VIIa signaling occurs via protease activated receptor 2. In a further aspect, the test compound is an antibody or small chemical entity. In a detailed aspect, the compound inhibits the binding of MAb 10H10 to tissue factor. In a further detailed aspect, the compound does not inhibit the binding of MAb 5G9 to tissue factor.

A method for treating inflammation in a mammalian subject is provided comprising administering a therapeutically effective amount of a compound which modulates signaling in cells via tissue factor-factor VIIa pathway, wherein said compound acts as an antagonist of tissue factor-factor VIIa signaling in a cell-based assay system, and said compound is effective to reduce or eliminate the disease or to prevent its occurrence or recurrence in the mammalian subject. In a further aspect, the compound does not interfere with hemostasis in the mammalian subject. In one aspect, tissue factor-factor VIIa signaling is dependent on protein disulfide isomerase. In another aspect, tissue factor-factor VIIa signaling occurs via protease activated receptor 2. In a further aspect, the test compound is an antibody or small chemical entity. In a detailed aspect, the compound inhibits the binding of MAb 10H10 to tissue factor. In a further detailed aspect, the compound does not inhibit the binding of MAb 5G9 to tissue factor.

A method for determining the presence of or predisposition to an angiogenesis-related disease state in a mammalian subject is provided comprising, providing a sample from the mammalian subject; introducing an antibody that binds immunospecifically to tissue factor in the sample, and determining the presence or amount of antibody bound to the tissue factor in the sample wherein the presence of antibody bound to the tissue factor is indicative of the presence of or predisposition to the angiogenesis-related disease state in the mammalian subject, wherein the antibody inhibits the tissue factor signaling and does not interfere with hemostasis in the mammalian subject. In a further aspect of the method, an increased level of antibody bound to the tissue factor indicates the presence of or predisposition to the angiogenesis-related disease state. In a detailed aspect, the antibody is Mab 10H10. In a further detailed aspect, the angiogenesis-related disease state is neoplastic disease or inflammation.

A method for determining the presence of or predisposition to a neoplastic disease state in a mammalian subject is provided comprising, providing a sample from the mammalian subject, introducing an antibody that binds immunospecifically to tissue factor in the sample, and determining the presence or amount of antibody bound to the tissue factor in the sample wherein the presence of antibody bound to the tissue factor is indicative of the presence of or predisposition to the neoplastic disease in the mammalian subject, wherein the antibody inhibits the tissue factor signaling and does not interfere with hemostasis in the mammalian subject. In a further aspect of the method, an increased level of antibody bound to the tissue factor indicates the presence of or predisposition to the neoplastic disease state. In a detailed aspect, the antibody is Mab 10H10. In a detailed aspect, the neoplastic disease state is solid tumor, benign or malignant breast cancer, melanoma, glioma, astrocytoma, hematological malignancy, leukemia, lung cancer, colorectal cancer, uterine cancer, uterine leiomyoma, ovarian cancer, endometrial cancer, polycystic ovary syndrome, endometrial polyps, prostate cancer, prostatic hypertrophy, pituitary cancer, adenomyosis, adenocarcinoma, meningioma, bone cancer, multiple myeloma, or CNS cancer.

A method for determining the presence of or predisposition to inflammatory disease in a mammalian subject is provided comprising, providing a sample from the mammalian subject; introducing an antibody that binds immunospecifically to tissue factor in the sample; and determining the presence or amount of antibody bound to the tissue factor in the sample wherein the presence of antibody bound to the tissue factor is indicative of the presence of or predisposition to the inflammatory disease in the mammalian subject, wherein the antibody inhibits the tissue factor signaling and does not interfere with hemostasis in the mammalian subject. In a further aspect of the method, an increased level of antibody bound to the tissue factor indicates the presence of or predisposition to the inflammatory disease state. In a detailed aspect, the antibody is Mab 10H10.

### BRIEF DESCRIPTION OF THE DRAWINGS

FIG. 1 shows specific inhibition of signaling tissue factor.

FIG. 2 shows signaling tissue factor is regulated by protein disulfide isomerase.

FIG. 3 shows signaling of reduced tissue factor.

FIG. 4 shows that TF-VIIa signaling promotes tumour growth.

FIG. 5 shows an epitope assignment for MAb-10H10.

FIG. 6 shows that inactivation of TF coagulant activity is dependent on nitric oxide.

FIG. 7 shows that TF-PAR2 complex formation is required for TF-VIIa signalling.

### DETAILED DESCRIPTION

In one aspect, the present invention provides compositions and methods for remedying abnormal tissue factor/factor VIIa (TF/VIIa) signaling activities (e.g., in subjects with excessive TF/VIIa signaling) and treating subjects suffering from diseases or conditions that are dependent upon, mediated by or associated with TF/VIIa signaling. Abnormal TF/VIIa signaling refers to excessive or insufficient activities of the tissue factor/factor VIIa (TF/VIIa) signaling pathway relative to that in healthy subjects. Diseases that are dependent upon TF/VIIa signaling encompass any disorders or condition the occurrence or development of which is mediated by or associated with abnormal signaling activities of the TF/VIIa pathway. Examples of such diseases include inflammation, neoplastic diseases, and angiogenesis-dependent diseases. Angiogenesis-dependent diseases encompass diseases or disorders with excessive angiogenesis (e.g., cancer, diabetic blindness, age-related macular degeneration, rheumatoid arthritis, and psoriasis) or insufficient angiogenesis (e.g., coronary artery disease, stroke, and delayed wound healing). Typically, the compositions of the invention comprise an inhibitor of TF/VIIa signaling pathway which does not interfere with TF-mediated hemostasis (e.g., coagulation) pathway. The methods of the invention comprise administering an effective amount of such an inhibitor to a mammalian subject in need of treatment. The inhibitor is effective in reducing the incidence of inflammation or neoplastic disease without increasing the risk of bleeding in the subject.

The enzymatic complex of the cell surface receptor tissue factor (TF) with the serine protease factor VIIa activates physiological haemostasis and coagulation and concomitantly triggers protease activated receptor signaling in inflammation, tumour progression and angiogenesis. Mackman, Arterioscler. Thromb. Vasc. Biol. 24: 1015-1022, 2004; Riewald and Ruf, Crit. Care 7: 123-129, 2003; Belting et al., Arterioscler. Thromb. Vasc. Biol. 25: 1545-1550, 2005. How direct upstream TF-VIIa signaling determines pathology without being overridden by abundantly generated downstream coagulation proteases remained a fundamental, unresolved question in vascular biology. As detailed in the Examples below, the present invention demonstrates that extracellular protein disulfide isomerase (PDI) associates with TF to regulate coagulation, while preserving cell signaling. Breaking of the extracellular TF Cys¹⁸⁶-Cys²⁰⁹ disulfide phenocopies functional properties of PDI-regulated signaling TF. Thus, disulfide exchange pathways act as extracellular switches for specificity of receptor function. A monoclonal antibody targeting the native conformation of signaling TF inhibited cellular responses and tumor growth in vivo (e.g., breast tumor or melanoma). The interruption of pathophysiological TF signaling without impairment of beneficial TF-induced haemostasis provides an example that functional disulfide switches can be exploited for therapeutic benefit.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Hemostasis” refers to the arrest of bleeding from an injured blood vessel, requires the combined activity of vascular, platelet, and plasma factors counterbalanced by regulatory mechanisms to limit the accumulation of platelets and fibrin in the area of injury. Hemostatic abnormalities can lead to thrombosis or excessive bleeding.

“Angiogenesis” refers to the growth of new blood vessels in a mammalian subject in either a healthy or disease state. Angiogenesis occurs during wound healing and to restore blood flow to tissues after injury or insult. In females, angiogenesis also occurs during the monthly reproductive cycle (to rebuild the uterus lining, to mature the egg during ovulation) and during pregnancy (to build the placenta, the circulation between mother and fetus). When angiogenic growth factors are produced in excess of angiogenesis inhibitors, blood vessel growth occurs. When inhibitors are present in excess of stimulators, angiogenesis ceases.

Excessive angiogenesis occurs in diseases including, but not limited to, cancer, inflammation, diabetic blindness, age-related macular degeneration, rheumatoid arthritis, or psoriasis. In these conditions, new blood vessels feed diseased tissues, destroy normal tissues, and in the case of cancer, allow tumor metastases. Insufficient angiogenesis occurs in diseases including, but not limited to, coronary artery disease, stroke, and delayed wound healing. In these conditions, inadequate blood vessels grow, and circulation is not properly restored, leading to the risk of tissue death.

### Treatment of Neoplastic Disease

Neoplastic disease refers to cancer or any malignant growth or tumor caused by abnormal and uncontrolled cell division; it may spread to other parts of the body through the lymphatic system or the blood stream. A “solid tumor” includes, but is not limited to, sarcoma, melanoma, carcinoma, or other solid tumor cancer.

“Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

“Melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

“Carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidemoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma viflosum.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leulcocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

### Treatment of Inflammation

“Inflammation” or “inflammatory disease” refers to both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

Protection of an animal from a disease involving inflammation refers to reducing the potential for an inflammatory response (i.e., a response involving inflammation) to an inflammatory agent (i.e., an agent capable of causing an inflammatory response, e.g., methacholine, histamine, an allergen, a leukotriene, saline, hyperventilation, exercise, sulfur dioxide, adenosine, propranolol, cold air, antigen and bradykinin). Preferably, the potential for an inflammatory response is reduced, optimally, to an extent that the animal no longer suffers discomfort and/or altered function from exposure to the inflammatory agent. For example, protecting an animal can refer to the ability of a compound, when administered to the animal, to prevent a disease from occurring and/or cure or alleviate disease symptoms, signs or causes. In particular, protecting an animal refers to modulating an inflammatory response to suppress (e.g., reduce, inhibit or block) an overactive or harmful inflammatory response. Also in particular, protecting an animal refers to regulating cell-mediated immunity and/or humoral immunity (i.e., T cell activity and/or IgE activity). Disease refers to any deviation from normal health of an animal and includes disease symptoms as well as conditions in which a deviation (e.g., infection, gene mutation, genetic defect, etc.) has occurred but symptoms are not yet manifested.

### Antibody Therapeutics

In some embodiments, the invention provides methods for inhibiting or suppressing TF/VIIa signaling in a mammalian subject that desires a reduced or down-regulated TF/VIIa signaling activities (e.g., one suffering from inflammation or tumor). The methods entail administering an inhibitor of TF/VIIa signaling to the mammalian subject in an amount effective to inhibit or suppress TF/VIIa signaling. In some related embodiments, the invention provides methods for treating or ameliorating the symptoms of diseases that are associated with or dependent upon tissue factor/factor VII signaling in a mammalian subject. As noted above, such diseases include, e.g., angiogenesis-related disease, neoplastic disease, or inflammation. The methods comprise administering an inhibitor of tissue factor signaling to the mammalian subject in an amount effective to reduce or eliminate the angiogenesis-related disease, neoplastic disease, or inflammation, or to prevent its occurrence or recurrence in the mammalian subject. In some embodiments, the invention utilizes an inhibitor which is an antibody to tissue factor that inhibits tissue factor signaling and which does not interfere with hemostasis (e.g., coagulation) in the mammalian subject.

A typical antibody refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

Antibodies and antibody-derived antigen-binding molecules denote polypeptide chain(s) which exhibit a strong monovalent, bivalent or polyvalent binding to a given epitope or epitopes (e.g., TF or the specific TF peptide epitope recognized by MAb 10H10). Unless otherwise noted, antibodies or antigen-binding molecules of the invention can have sequences derived from any vertebrate, camelid, avian or pisces species. They can be generated using any suitable technology hybridoma technology, ribosome display, phage display, gene shuffling libraries, semi-synthetic or fully synthetic libraries or combinations thereof. As detailed herein, antibodies or antigen-binding molecules of the invention include intact antibodies, antigen-binding polypeptide chains and other designer antibodies (see, e.g., Serafini, J Nucl Med. 34:533-6, 1993).

Antibody or antigen-binding molecule also includes antibody fragments which contain the antigen-binding portions of an intact antibody that retain capacity to bind the cognate antigen (e.g., TF or the specific TF peptide epitope recognized by MAb 10H10). Examples of such antibody fragments include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242:423-426, 1988; and Huston et al., Proc. Natl. Acad. Sci. USA 85:5879-5883, 1988.

Antibodies or antigen-binding molecules of the invention further include one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies.

The various antibodies or antigen-binding fragments described herein can be produced by enzymatic or chemical modification of the intact antibodies, or synthesized de novo using recombinant DNA methodologies (e.g., single chain Fv), or identified using phage display libraries (see, e.g., McCafferty et al., Nature 348:552-554, 1990). For example, minibodies can be generated using methods described in the art, e.g., Vaughan and Sollazzo, Comb Chem High Throughput Screen. 4:417-30 2001. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Single chain antibodies can be identified using phage display libraries or ribosome display libraries, gene shuffled libraries. Such libraries can be constructed from synthetic, semi-synthetic or nave and immunocompetent sources.

One specific example of antibodies that can be employed in the practice of the therapeutic methods noted above is the murine monoclonal antibody designated 10H10. As demonstrated in the Examples below, MAb 10H10 is an antibody that acts as an inhibitor of tissue factor signaling without interfering with hemostasis. This antibody has been described in great detail in U.S. Pat. Nos. 5,223,427 and 6,001,978. Hybridoma secreting this antibody has been deposited pursuant to Budapest Treaty requirements with the American Type Culture Collection (ATCC) (Manassas, Va.) on Mar. 27, 1987 with accession number HB9383. In addition to the 10H10 antibody produced by this hybridoma, any antibody which has the same binding specificity and the same or better binding affinity of MAb 10H10 can also be used in the therapeutic methods of the invention. In addition, the therapeutic methods of the invention can also use any antigen-binding molecule or fragments that are derived from MAb 10H10 or an antibody with the same binding specificity and the same or better binding affinity of MAb 10H10.

Some of the therapeutic methods of the invention are directed to treating human subjects. In these methods, a humanized antibody, a human antibody, or a chimeric antibody containing human sequences (e.g., in the constant region) is preferred. Compared to an antibody isolated from a non-human animal (e.g., a mouse), such an antibody would have less or no antigenicity when administered to the human subject. A chimeric anti-TF antibody (e.g., one with the same binding specificity as that of MAb 10H10) can be made up of regions from a non-human anti-TF antibody together with regions of human antibodies. For example, a chimeric H chain can comprise the antigen binding region of the heavy chain variable region of a mouse anti-TF antibody exemplified herein linked to at least a portion of a human heavy chain constant region. This chimeric heavy chain may be combined with a chimeric L chain that comprises the antigen binding region of the light chain variable region of the mouse anti-TF antibody linked to at least a portion of the human light chain constant region.

Chimeric anti-TF antibodies of the invention can be produced in accordance with methods known in the art. See, e.g., Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science 240:1041-1043, 1988; Liu et al., PNAS 84:3439-3443, 1987; Liu et al., J. Immunol. 139:3521-3526, 1987; Sun et al., PNAS 84:214-218, 1987; Nishimura et al., Canc. Res. 47:999-1005, 1987; Wood et al., Nature 314:446-449, 1985; Shaw et al., J. Natl. Cancer Inst. 80:1553-1559, 1988.

Chimeric antibodies which have the entire variable regions from a non-human antibody can be further humanized to reduce antigenicity of the antibody in human. This is typically accomplished by replacing certain sequences or amino acid residues in the Fv variable regions (framework regions or non-CDR regions) with equivalent sequences or amino acid residues from human Fv variable regions. These additionally substituted sequences or amino acid residues are usually not directly involved in antigen binding. More often, humanization of a non-human antibody proceeds by substituting only the CDRs of a non-human antibody (e.g., the mouse anti-TF antibodies exemplified herein) for the CDRs in a human antibody. In some cases, this is followed by replacing some additional residues in the human framework regions with the corresponding residues from the non-human donor antibody. Such additional grafting is often needed to improve binding to the antigen. This is because humanized antibodies which only have CDRs grafted from a non-human antibody can have less than perfect binding activities as compared to that of the non-human donor antibody. Thus, in addition to the CDRs, humanized anti-hTF antibodies of the invention (e.g., one with the same binding specificity as that of MAb 10H10) can often have some amino acids residues in the human framework region replaced with corresponding residues from the non-human donor antibody (e.g., the mouse antibody exemplified herein). Methods for generating humanized antibodies by CDR substitution, including criteria for selecting framework residues for replacement, are well known in the art. See, e.g., Winter et al., UK Patent Application GB 2188638A (1987), U.S. Pat. No. 5,225,539; Jones et al., Nature 321:552-525, 1986; Verhoeyan et al., Science 239:1534, 1988; Beidler et al., J. Immunol. 141:4053-4060, 1988; and WO 94/10332.

In addition to chimeric or humanized anti-hPAR1 antibodies, therapeutic methods for treating human subjects can also employ fully human antibodies that exhibit the same binding specificity and comparable or better binding affinity relative to a mouse antibody such as MAb 10H10. The human anti-TF antibodies can be generated using any of the methods that are well known in the art, e.g., phage display methods using antibody libraries derived from human immunoglobulin sequences. See, e.g., Lonberg and Huszar, Int. Rev. Immunol. 13:65-93 (1995), U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Derivative antibodies or antigen-binding molecules which have the same binding specificity and the same or better binding affinity of MAb 10H10 can be obtained by methods well known in the art and exemplified herein. For example, candidate antibodies or immunoglobulins generated against a tissue factor antigen can be screened for, e.g., an ability to compete with MAb 10H10 for binding to a tissue factor polypeptide or peptide. Polypeptide and polynucleotide sequences of human tissue factor are known (see, e.g., Scarpati et al., Biochemistry 26:5234-5238, 1987; and Fisher et al., Thromb. Res. 48:89-99, 1987). Tissue factor polypeptides or peptides suitable for the screening can be generated using methods well known in the art or described herein. In addition, a panel of specific antigenic peptides derived from human tissue factor have been described in the art, e.g., U.S. Pat. Nos. 5,223,427 and 6,001,978. These patents also disclose the profile of MAb 10H10 binding to the panel of tissue factor peptides. For example, it was shown that MAb 10H10 specifically binds to tissue factor peptide with the sequence of SGTTNTVAAYNLTWKSTNFKTILEWEPKPV (SEQ ID NO:1) or ECDLTDEIVKDVKQTY (SEQ ID NO:2) but not several other antigenic peptides derived from human tissue factor. The latter peptides include, e.g., TKSGDWKSKCFYTTDTECDLTDEIVKDVKQTY (SEQ ID NO:3) or LARVFSYPAGNVESTGSAGEPLYENSPEFTPYLC (SEQ ID NO:4). Thus, candidate antibodies (e.g., antibodies generated against a human tissue factor polypeptide) can be screened for ability to block MAb 10H10 binding to the peptide with the sequence of SEQ ID NO:1 and/or SEQ ID NO:2. The can also be screened for the same or substantially identical binding profile as that of MAb 10H10 for binding to the panel of human tissue factor peptides as described in U.S. Pat. No. 5,223,427. Methods for performing such screening is well known in the art (see, e.g., U.S. Pat. Nos. 5,223,427 and 6,001,978) and also described herein.

In addition in vitro screening assays, in vivo methods can also be used to identify anti-TF antibodies that are suitable for practicing the methods of the present invention. For example, an in vivo method for replacing a nonhuman antibody variable region with a human variable region in an antibody while maintaining the same or providing better binding characteristics has been disclosed in U.S. patent application Ser. No. 10/778,726 (Publication No. 20050008625). To generate a human antibody with the same binding specificity and the same or better binding affinity as that of mouse MAb 10H10, this method relies on epitope guided replacement of variable regions of the non-human antibody with a fully human antibody. The resulting human antibody is generally unrelated structurally to the reference nonhuman antibody, but binds to the same epitope on the same antigen as the reference antibody.

Human antibodies with the same or better affinities for a specific epitope than a starting non-human antibody (e.g., a mouse MAb 10H10) can also be obtained from companies which customarily produce human antibodies. For example, to generate a desired human antibody, KaloBios, Inc. (Mountain View, Calif.) employs a human “acceptor” antibody library. A directed or epitope focused library of human antibodies which bind to the identical epitope as the non-human antibody, though with varying affinities, is then generated. Antibodies in the epitope focused library are then selected for similar or higher affinity than that of the starting non-human antibody. The identified human antibodies are then subject to further analysis for affinity and sequence identity.

In addition to MAb 10H10 and antibodies or antigene-binding molecules derived therefrom, other anti-TF antibodies with the desired properties in order to practice the therapeutic methods of the invention can also be readily produced using techniques and methods that are routinely practiced in the art. As is well understood in the art, biospecific capture reagents include antibodies, binding fragments of antibodies which bind to tissue factor, e.g., on metastatic cells or inflammatory cells (e.g., single chain antibodies, Fab′ fragments, F(ab)′2 fragments, and scFv proteins and affibodies (Affibody, Teknikringen 30, floor 6, Box 700 04, Stockholm SE-10044, Sweden; See U.S. Pat. No. 5,831,012, incorporated herein by reference in its entirety and for all purposes)). Depending on intended use, they also may include receptors and other proteins that specifically bind another biomolecule.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See, for example, U.S. Application No. 20030022244.

Initially, a predetermined target object is chosen to which an antibody may be raised. Techniques for generating monoclonal antibodies directed to target objects are well known to those skilled in the art. Examples of such techniques include, but are not limited to, those involving display libraries, xeno or humab mice, hybridomas, and the like. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

Antibodies (Abs) that can be subjected to the techniques set forth herein include monoclonal and polyclonal Abs, and antibody fragments such as Fab, Fab′, F(ab′)₂, Fd, scFv, diabodies, antibody light chains, antibody heavy chains and/or antibody fragments derived from phage or phagemid display technologies. To begin with, an initial antibody is obtained from an originating species. More particularly, the nucleic acid or amino acid sequence of the variable portion of the light chain, heavy chain or both, of an originating species antibody having specificity for a target antigen is needed. The originating species is any species which was used to generate the antibodies or antibody libraries, e.g., rat, mice, rabbit, chicken, monkey, human, and the like Techniques for generating and cloning monoclonal antibodies are well known to those skilled in the art. After a desired antibody is obtained, the variable regions (V_{H} and V_{L}) are separated into component parts (i.e, frameworks (FRs) and CDRs) using any possible definition of CDRs (e.g., Kabat alone, Chothia alone, Kabat and Chothia combined, and any others known to those skilled in the art). Once that has been obtained, the selection of appropriate target species frameworks is necessary. One embodiment involves alignment of each individual framework region from the originating species antibody sequence with variable amino acid sequences or gene sequences from the target species. Programs for searching for alignments are well known in the art, e.g., BLAST and the like. For example, if the target species is human, a source of such amino acid sequences or gene sequences (germline or rearranged antibody sequences) may be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain embodiments, an acceptable range of homology is greater than about 50%. It should be understood that target species may be other than human.

“Treating” refers to any indicia of success in the treatment or amelioration or prevention of an cancer or inflammation, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with ocular disease. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Treating” or “treatment” of cancer, metastatic cancer or inflammation using the methods of the present invention includes preventing the onset of symptoms in a subject that may be at increased risk of cancer or inflammation but does not yet experience or exhibit symptoms of infection, inhibiting the symptoms of cancer or inflammation (slowing or arresting its development), providing relief from the symptoms or side-effects of cancer or inflammation (including palliative treatment), and relieving the symptoms of cancer or inflammation (causing regression). “Treating” refers to any indicia of success in the treatment or amelioration or prevention of an cancer or inflammation, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with ocular disease. The term “therapeutic effect” refers to the reduction, elimination, or prevention of the disease, symptoms of the disease, or side effects of the disease in the subject.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

The terms “identical” or percent “identity,” in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding tissue factor or antibody to tissue factor described herein or amino acid sequence of a tissue factor or antibody to tissue factor described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math, 2: 482, 1981, by the homology alignment algorithm of Needleman and Wunsch, J. Mol. Biol, 48:443, 1970, by the search for similarity method of Pearson and Lipman, Proc. Nat'l. Acad. Sci. USA, 85:2444, 1988, by computerized implementations of these algorithms (GAP, BESTFIT, FASTA, and TFASTA in the Wisconsin Genetics Software Package, Genetics Computer Group, 575 Science Dr. Madison, Wis.), or by manual alignment and visual inspection (see, e.g. Ausubel et al., eds., Current Protocols in Molecular Biology. 1995 supplement).

A preferred example of algorithm that is suitable for determining percent sequence identity and sequence similarity are the BLAST and BLAST 2.0 algorithms, which are described in Altschul et al., Nuc. Acids Res, 25:3389-3402, 1977 and Altschul et al., J. Mol. Biol, 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA, 89:10915, 1989) alignments (B) of 50, expectation (E) of 10, M=5, N=−4, and a comparison of both strands.

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell, 3rd ed., 1994) and Cantor and Schimmel, Biophysical Chemistry Part I: The Conformation of Biological Macromolecules, 1980. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, “Techniques in Biochemistry and Molecular Biology—Hybridization with Nucleic Probes,” Overview of principles of hybridization and the strategy of nucleic acid assays, 1993. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications, Academic Press, Inc. N.Y., 1990.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

“Pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compositions of the invention can be administered. In some embodiments of the present invention, the patient will be suffering from a condition that causes lowered resistance to disease, e.g., HIV. In an exemplary embodiment of the present invention, to identify subject patients for treatment with a pharmaceutical composition comprising one or more collectins and/or surfactant proteins according to the methods of the invention, accepted screening methods are employed to determine the status of an existing disease or condition in a subject or risk factors associated with a targeted or suspected disease or condition. These screening methods include, for example, ocular examinations to determine whether a subject is suffering from an ocular disease. These and other routine methods allow the clinician to select subjects in need of therapy. In certain embodiments of the present invention, ophthalmic compositions for storing, cleaning, re-wetting and/or disinfecting a contact lens, as well as artificial tear compositions and/or contact lenses will contain one or more collectins and/or surfactant proteins thereby inhibiting the development of ocular disease in contact-lens wearers.

“Concomitant administration” of a known cancer therapeutic drug or inflammation therapeutic drug with a pharmaceutical composition of the present invention means administration of the drug and the composition which is an inhibitor of tissue factor, e.g., antibody or small chemical entity, at such time that both the known drug and the composition of the present invention will have a therapeutic effect. Such concomitant administration may involve concurrent (i.e. at the same time), prior, or subsequent administration of the antimicrobial drug with respect to the administration of a compound of the present invention. A person of ordinary skill in the art, would have no difficulty determining the appropriate timing, sequence and dosages of administration for particular drugs and compositions of the present invention.

After selecting suitable frame work region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) may be produced by oligonucleotide synthesis and/or PCR. The nucleic acid encoding CDR regions may also be isolated from the originating species antibodies using suitable restriction enzymes and ligated into the target species framework by ligating with suitable ligation enzymes. Alternatively, the framework regions of the variable chains of the originating species antibody may be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

Assembly of a physical antibody or antibody fragment library is preferably accomplished using synthetic oligonucleotides. In one example, oligonucleotides are designed to have overlapping regions so that they could anneal and be filled in by a polymerase, such as with polymerase chain reaction (PCR). Multiple steps of overlap extension are performed in order to generate the V_{L} and V_{R} gene inserts. Those fragments are designed with regions of overlap with human constant domains so that they could be fused by overlap extension to produce full length light chains and Fd heavy chain fragments. The light and heavy Fd chain regions may be linked together by overlap extension to create a single Fab library insert to be cloned into a display vector. Alternative methods for the assembly of the humanized library genes can also be used. For example, the library may be assembled from overlapping oligonucleotides using a Ligase Chain Reaction (LCR) approach. Chalmers et al., Biotechniques, 30-2: 249-252, 2001.

Various forms of antibody fragments may be generated and cloned into an appropriate vector to create a hybrid antibody library or hybrid antibody fragment library. For example variable genes can be cloned into a vector that contains, in-frame, the remaining portion of the necessary constant domain. Examples of additional fragments that can be cloned include whole light chains, the Fd portion of heavy chains, or fragments that contain both light chain and heavy chain Fd coding sequence. Alternatively, the antibody fragments used for humanization may be single chain antibodies (scFv).

Any selection display system may be used in conjunction with a library according to the present disclosure. Selection protocols for isolating desired members of large libraries are known in the art, as typified by phage display techniques. Such systems, in which diverse peptide sequences are displayed on the surface of filamentous bacteriophage have proven useful for creating libraries of antibody fragments (and the nucleotide sequences that encode them) for the in vitro selection and amplification of specific antibody fragments that bind a target antigen. Scott et al., Science, 249: 386, 1990. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage or T7 capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward. Methods for the construction of bacteriophage antibody display libraries and lambda phage expression libraries are well known in the art. McCafferty et al, Nature, 348: 552, 1990; Kang et al., Proc. Nat. Acad. Sci. U.S.A., 88: 4363, 1991.

The present invention further relates to antibodies and T-cell antigen receptors (TCR) which specifically bind the polypeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding molecules or fragments, including single-chain antibodies, may comprise the variable region(s) alone or in combination with the entire or partial of the following: hinge region, CH₁, CH₂, and CH₃ domains. Also included in the invention are any combinations of variable region(s) and hinge region, CH₁, CH₂, and CH₃ domains. The present invention further includes monoclonal, polyclonal, chimeric, humanized, and human monoclonal and human polyclonal antibodies which specifically bind the polypeptides of the present invention. The present invention further includes antibodies which are anti-idiotypic to the antibodies of the present invention.

As noted above, antibodies suitable for the present invention may be monospecific, bispecific, trispecific or of greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt et al., J. Immunol. 147: 60-69, 1991; U.S. Pat. Nos. 5,573,920; 4,474,893; 5,601,819; 4,714,681; 4,925,648, each incorporated herein by reference in their entirety and for all purposes; Kostelny et al., J. Immunol. 148: 1547-1553, 1992.

Antibodies suitable for the present invention may be described or specified in terms of the epitope(s) or portion(s) of a polypeptide of the present invention which are recognized or specifically bound by the antibody. The epitope(s) or polypeptide portion(s) may be specified as described herein, e.g., by N-terminal and C-terminal positions, by size in contiguous amino acid residues. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention may also be described or specified in terms of their binding affinity. Preferred binding affinities include those with a dissociation constant or K_{d} less than 5×10⁻⁶M, 10⁻⁶M, 5×10⁻⁷M, 10⁻⁷M, 5×10⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies that inhibit tissue factor signaling have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow and Lane, supra, incorporated herein by reference in its entirety and for all purposes.

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387, each incorporated herein by reference in their entirety and for all purposes.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not a limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art and taught in Harlow and Lane, supra; Hammerling et al., Monoclonal Antibodies and T-Cell Hybridomas, 563-681, 1981, said references incorporated by reference in their entireties. Fab and F(ab′)₂ fragments may be produced by proteolytic cleavage, using enzymes such as papain (to produce Fab fragments) or pepsin (to produce F(ab′)₂ fragments).

Alternatively, antibodies that inhibit tissue factor signaling can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182: 41-50, 1995; Ames et al., J. Immunol. Methods 184: 177-186, 1995; Kettleborough et al., Eur. J. Immunol. 24: 952-958, 1994; Persic et al., Gene 187: 9-18, 1997; Burton et al, Advances in Immunology 57:191-280, 1994; PCT/GB91/01134; WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727 and 5,733,743, each incorporated herein by reference in their entirety and for all purposes.

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)2 fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Examples of techniques which can be used to produce single-chain Fvs and antibodies include those described in U.S. Pat. Nos. 4,946,778 and 5,258,498, each incorporated herein by reference in their entirety and for all purposes; Huston et al., Methods in Enzymology, 203: 46-88, 1991; Shu, L. et al., PNAS 90: 7995-7999, 1993; and Skerra et al., Science 240: 1038-1040, 1988. For some uses, including in vivo use of antibodies in humans and in vitro detection assays, it may be preferable to use chimeric, humanized, or human antibodies. Methods for producing chimeric antibodies are known in the art. See e.g., Morrison, Science 229: 1202, 1985; Oi et al., BioTechniques 4: 214, 1986; Gillies et al., J. Immunol. Methods, 125: 191-202, 1989; and U.S. Pat. No. 5,807,715. Antibodies can be humanized using a variety of techniques including CDR-grafting (EP 0 239 400; WO 91/09967; and U.S. Pat. Nos. 5,530,101 and 5,585,089), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). Human antibodies can be made by a variety of methods known in the art including phage display methods described above. See also, U.S. Pat. Nos. 4,444,887; 4,716,111; 5,545,806; and 5,814,318; and WO 98/46645; WO 98/50433; WO 98/24893; WO 98/16654; WO 96/34096; WO 96/33735; and WO 91/10741, each incorporated herein by reference in their entirety and for all purposes.

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and WO 93/21232; EP 0 439 095; Naramura et al., Immunol. Lett. 39: 91-99, 1994; U.S. Pat. No. 5,474,981, incorporated herein by reference in its entirety and for all purposes; Gillies et al., PNAS 89: 1428-1432, 1992; Fell et al., J. Immunol. 146: 2446-2452, 1991.

The present invention further includes compositions comprising the polypeptides of the present invention fused or conjugated to antibody domains other than the variable regions. For example, the polypeptides of the present invention may be fused or conjugated to an antibody Fe region, or portion thereof. The antibody portion fused to a polypeptide of the present invention may comprise the hinge region, CH₁ domain, CH₂ domain, and CH₃ domain or any combination of whole domains or portions thereof. The polypeptides of the present invention may be fused or conjugated to the above antibody portions to increase the in vivo half life of the polypeptides or for use in immunoassays using methods known in the art. The polypeptides may also be fused or conjugated to the above antibody portions to form multimers. For example, Fe portions fused to the polypeptides of the present invention can form dimers through disulfide bonding between the Fe portions. Higher multimeric forms can be made by fusing polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 0 307 434, EP 0 367 166; WO 96/04388; and WO 91/06570, each incorporated herein by reference in their entirety and for all purposes; Ashkenazi et al., PNAS, 88: 10535-10539, 1991; Zheng et al., J. Immunol., 154: 5590-5600, 1995; and Vil et al., PNAS, 89: 11337-11341, 1992.

The invention further relates to antibodies which act as antagonists of tissue factor signaling in the present invention. For example, the present invention includes antibodies which disrupt the receptor/ligand interactions with the unique conformation of the polypeptides of the invention either partially or fully. Included are both receptor-specific antibodies and ligand-specific antibodies. Included are receptor-specific antibodies which do not prevent ligand binding but prevent receptor signaling. Receptor signaling may be determined by techniques described herein or otherwise known in the art. Also include are receptor-specific antibodies which both prevent ligand binding and receptor signaling. Likewise, included are neutralizing antibodies which bind the ligand and prevent binding of the ligand to the receptor, as well as antibodies which bind the ligand, thereby preventing receptor signaling, but do not prevent the ligand from binding the receptor. Further included are antibodies which activate the receptor. These antibodies may act as antagonists for either all or less than all of the biological activities affected by ligand-mediated receptor signaling. The antibodies may be specified as antagonists for biological activities comprising specific activities disclosed herein. The above antibody antagonists can be made using methods known in the art. See e.g., WO 96/40281; U.S. Pat. No. 5,811,097, each incorporated herein by reference in their entirety and for all purposes; Deng et al., Blood 92: 1981-1988, 1998; Chen, et al., Cancer. Res., 58: 3668-3678, 1998; Harrop et al., J. Immunol. 161: 1786-1794, 1998; Zhu et al., Cancer Res., 58: 3209-3214, 1998; Yoon, et al., J. Immunol., 160: 3170-3179, 1998; Prat et al., J. Cell. Sci., 111: 237-247, 1998; Pitard et al., J. Immunol. Methods, 205: 177-190, 1997; Liautard et al., Cytokinde, 9: 233-241, 1997; Carlson et al., J. Biol. Chem., 272: 11295-11301, 1997; Taryman et al., Neuron, 14: 755-762, 1995; Muller et al., Structure, 6: 1153-1167, 1998; Bartunek et al., Cytokinem, 8: 14-20, 1996. As discussed above, antibodies to that inhibit tissue factor signaling on neoplastic cells or inflammatory cells can, in turn, be utilized to generate anti-idiotype antibodies that “mimic” polypeptides of the invention using techniques well known to those skilled in the art. (See, e.g., Greenspan et al., FASEB J. 7: 437-444, 1989 and Nissinoff, J. Immunol. 147: 2429-2438, 1991). For example, antibodies which bind to and competitively inhibit polypeptide multimerization and/or binding of a polypeptide of the invention to ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

“Inhibitors,” “activators,” and “modulators” of tissue factor signaling on neoplastic cells or inflammatory cells are used to refer to inhibitory, activating, or modulating molecules, respectively, identified using in vitro and in vivo assays for tissue factor binding or signaling, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate the activity of signaling tissue factor, e.g., antagonists. Activators are agents that, e.g., bind to, stimulate, increase, open, activate, facilitate, enhance activation, sensitize or up regulate the activity of signaling tissue factor, e.g., agonists. Modulators include agents that, e.g., alter the interaction of signaling tissue factor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring signaling tissue factor, e.g., with altered activity, as well as naturally occurring and synthetic ligands, antagonists, agonists, small chemical molecules and the like. Such assays for inhibitors include, e.g., applying putative modulator compounds to a cell expressing signaling tissue factor and then determining the functional effects on tissue factor signaling, as described herein. Samples or assays comprising tissue factor that is treated with a potential activator, inhibitor, or modulator are compared to control samples without the inhibitor, activator, or modulator to examine the extent of inhibition. Control samples (untreated with inhibitors) can be assigned a relative tissue factor signaling value of 100%. Inhibition is achieved when the tissue factor signaling activity value relative to the control is about 80%, optionally 50% or 25-0%.

The ability of a molecule to bind to signaling tissue factor can be determined, for example, by the ability of the putative ligand to bind to signaling tissue factor on cells. Specificity of binding can be determined by comparing binding to cells that only have coagulation tissue factor.

In one embodiment, antibody binding to signaling tissue factor can be assayed by either immobilizing the ligand or the receptor. For example, the assay can include immobilizing tissue factor appropriately modified to mimic the signaling conformation fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

### Fusion Proteins

Antibodies to signaling tissue factor can be used to generate fusion proteins. For example, the antibodies of the present invention, when fused to a second protein, can be used as an antigenic tag for purification of the antibody or to increase stability of the antibody as a therapeutic treatment as an inhibitor of signaling tissue factor.

Examples of domains that can be fused to polypeptides include not only heterologous signal sequences, but also other heterologous functional regions. The fusion does not necessarily need to be direct, but may occur through linker sequences.

Moreover, fusion proteins may also be engineered to improve characteristics of the polypeptide. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides only requires familiar and routine techniques in the art.

Moreover, antibody compositions and compositions that inhibit tissue factor signaling, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. EP A 394,827; Traunecker et al., Nature, 331: 84-86, 1988. Fusion proteins having disulfide-linked dimeric structures (due to the IgG) can also be more efficient in binding and neutralizing other molecules, than the monomeric secreted protein or protein fragment alone. Fountoulakis et al., J. Biochem. 270: 3958-3964, 1995.

Similarly, EP-A-O 464 533 (Canadian counterpart 2045869) discloses fusion proteins comprising various portions of constant region of immunoglobulin molecules together with another human protein or part thereof. In many cases, the Fe part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. Bennett et al., J. Molecular Recognition 8: 52-58, 1995; K. Johanson et al., J. Biol. Chem., 270: 9459-9471 1995.

Moreover, the polypeptides can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusions can be engineered using the polynucleotides or the polypeptides of the present invention.

scFv Phage Libraries

A library of scFv antibodies that inhibit tissue factor signaling in a mammalian subject and which does not interfere with hemostasis in the mammalian subject can be used to treat a angiogenesis, neoplastic disease or inflammatory disease. One approach for a phage display library to identify an antibody composition that specifically binds to and inhibits signaling tissue factor but does not increase the risk of bleeding, has been the use of scFv phage-libraries (see, e.g., Huston et al., Proc. Natl. Acad. Sci. U.S.A., 85: 5879-5883, 1988; Chaudhary et al., Proc. Natl. Acad. Sci. U.S.A., 87: 1066-1070, 1990. Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference. The display of Fab libraries is also known, for instance as described in WO92/01047 (CAT/MRC) and WO91/17271 (Affymax).

Hybrid antibodies or hybrid antibody fragments that are cloned into a display vector can be selected that inhibit tissue factor signaling for treatment of a neoplastic disease or inflammatory disease in order to identify variants that maintained good binding activity because the antibody or antibody fragment will be present on the surface of the phage or phagemid particle. See for example Barbas III, et al., Phage Display, A Laboratory Manual, Cold Spring Harbor Laboratory Press, Cold Spring Harbor, N.Y., 2001, the contents of which are incorporated herein by reference. For example, in the case of Fab fragments, the light chain and heavy chain Fd products are under the control of a lac promoter, and each chain has a leader signal fused to it in order to be directed to the periplasmic space of the bacterial host. It is in this space that the antibody fragments will be able to properly assemble. The heavy chain fragments are expressed as a fusion with a phage coat protein domain which allows the assembled antibody fragment to be incorporated into the coat of a newly made phage or phagemid particle. Generation of new phagemid particles requires the addition of helper phage which contain all the necessary phage genes. Once a library of antibody fragments is presented on the phage or phagemid surface, a process termed panning follows. This is a method whereby i) the antibodies displayed on the surface of phage or phagemid particles are bound to the desired antigen, ii) non-binders are washed away, iii) bound particles are eluted from the antigen, and iv) eluted particles are exposed to fresh bacterial hosts in order to amplify the enriched pool for an additional round of selection. Typically three or four rounds of panning are performed prior to screening antibody clones for specific binding. In this way phage/phagemid particles allow the linkage of binding phenotype (antibody) with the genotype (DNA) making the use of antibody display technology very successful. However, other vector formats could be used for this humanization process, such as cloning the antibody fragment library into a lytic phage vector (modified T7 or Lambda Zap systems) for selection and/or screening.

After selection of desired hybrid antibodies and/or hybrid antibody fragments, it is contemplated that they can be produced in large volume by any technique known to those skilled in the art, e.g., prokaryotic or eukaryotic cell expression and the like. For example, hybrid antibodies or fragments may be produced by using conventional techniques to construct an expression vector that encodes an antibody heavy chain in which the CDRs and, if necessary, a minimal portion of the variable region framework, that are required to retain original species antibody binding specificity (as engineered according to the techniques described herein) are derived from the originating species antibody and the remainder of the antibody is derived from a target species immunoglobulin which may be manipulated as described herein, thereby producing a vector for the expression of a hybrid antibody heavy chain.

In a detailed embodiment, a single-chain Fv (scFv) antibody library can be prepared from the peripheral blood lymphocytes of 5, 10, 15, or 20 or more patients with various cancer diseases. Completely human high-affinity scFv antibodies can then be selected by using synthetic sialyl Lewis^{x} and Lewis^{x} BSA conjugates. In one study, these human scFv antibodies were specific for sialyl Lewis^{x} and Lewis^{x}, as demonstrated by ELISA, BIAcore, and flow cytometry binding to the cell surface of pancreatic adenocarcinoma cells. Nucleotide sequencing revealed that at least four unique scFv genes were obtained. The K_{d} values ranged from 1.1 to 6.2×10⁻⁷ M that were comparable to the affinities of mAbs derived from the secondary immune response. These antibodies could be valuable reagents for probing the structure and function of carbohydrate antigens and in the treatment of human tumor diseases. Mao, et al., Proc. Natl. Acad. Sci. U.S.A. 96: 6953-6958, 1999.

In a further detailed embodiment, phage displayed combinatorial antibody libraries can be used to generate and select a wide variety of antibodies to an appropriate antigen associated, e.g., antibodies that inhibit tissue factor signaling for treatment of a neoplastic disease or inflammatory disease. The phage coat proteins pVII and pIX can be used to display the heterodimeric structure of the antibody Fv region. Aspects of this technology have been extended to construct a large, human single-chain Fv (scFv) library of 4.5×10⁹ members displayed on pIX of filamentous bacteriophage. Furthermore, the diversity, quality, and utility of the library were demonstrated by the selection of scFv clones against six different protein antigens. Notably, more than 90% of the selected clones showed positive binding for their respective antigens after as few as three rounds of panning. Analyzed scFvs were also found to be of high affinity. For example, kinetic analysis (BIAcore) revealed that scFvs against staphylococcal enterotoxin B and cholera toxin B subunit had a nanomolar and subnanomolar dissociation constant, respectively, affording affinities comparable to, or exceeding that, of mAbs obtained from immunization. High specificity was also attained, not only between very distinct proteins, but also in the case of more closely related proteins, e.g., Ricinus communis (“ricin”) agglutinins (RCA₆₀ and RCA₁₂₀), despite >80% sequence homology between the two. The results suggested that the performance of pIX-display libraries can potentially exceed that of the pIII-display format and make it ideally suited for panning a wide variety of target antigens. Gao et al., Proc. Natl. Acad. Sci. U.S.A. 99: 12612-12616, 2001.

Specific binding between an antibody or other binding agent and an antigen means a binding affinity of at least 10⁻⁶ M. Preferred binding agents bind with affinities of at least about 10⁻⁷ M, and preferably 10⁻⁸ M to 10⁻⁹ M, 10⁻¹⁰M, 10⁻¹¹M, or 10⁻¹² M. The term epitope means an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

### Immunological Binding Assays for Detection of Tissue Factor Signaling and Modulators Thereof

The invention provide methods for detecting tissue factor signaling and for identifying modulators of tissue factor signaling. As described in more detail below, some of the methods are directed to identifying modulators of tissue factor signaling in a cell-based assay system. Some other methods are directed to identifying modulators of tissue factor signaling in a cell-free system. In some embodiments, test compounds are screened to identify tissue factor modulators which inhibit or suppress TF/VIIa mediated signaling activities but do not interfere with hemostasis in vivo. Such modulators (e.g., small molecule organic compound modulators) can be identified by employing a known compound that possesses such desired properties (e.g., MAb 10H10) in the various competitive assay formats described herein. Thus, some of the screening methods of the invention are directed to identifying compounds which inhibit TF/VIIa signaling but does not block coagulation. These methods entail measuring in the presence or absence of test compounds a binding between (i) an antibody or an antigen-binding molecule having the binding specificity of MAb 10H10 and (ii) a tissue factor polypeptide, and then detecting an inhibition of the binding in the presence of a test compound relative to the binding in the absence of the test compound. Some of these methods employ the murine MAb 10H10 produced by the hybridoma with ATCC access number HB9383. Some of the screening methods employ test compounds which are preferably small molecule organic compounds, e.g., chemical compounds with a molecular weight of not more than about 5000, and more preferably not more than about 2,500, 1,000 or 500.

Any of the techniques and assay formats described herein can be used to practice these methods. In addition to measuring their ability to compete with MAb 10H10 for binding to tissue factor, the modulators thus identified can be additionally examined for activity to modulate tissue factor signaling (e.g., inhibiting TF/VIIa signaling activities while having no significant effect on hemostasis). The compounds can be tested for inhibitory activity on any of the signaling activities that are mediated by TF/VIIa as described herein (e.g., MAP kinase phosphorylation or complex formation with and signaling via protease activated receptor 2). Assays for measuring TF/VIIa mediated signaling activities are well known in the art. As exemplified in Example 8 below, TF/VIIa mediated signaling activities can be quantitatively measured by a MAP kinase phosphorylation assay, e.g., assaying by western blot phosphorylation level of a MAP kinase (e.g., ERK kinase) in HUVEC cells or CHO cells stimulated with factors VIIa and X. These assays can be used in the screening methods described herein for identifying novel modulating compounds (e.g., inhibitors) of TF signalling. They can also be used in the therapeutic methods of the invention to monitor the effect of an employed inhibitor of TF signaling. A compound is considered a TF signaling inhibitor if the compound can inhibit TF signaling activities by at least 50%, at least 75%, at least 90%, or at least 95% relative to TF signaling in the absence of the compound. The quantitative inhibition can be measured by any of the TF signaling assays well known in the art (see, e.g., Ahamed et al., Blood 105:2384-91, 2005) or described herein, e.g., a reduction of ERK phosphorylation level in HUVEC cells over a 6-day period under the conditions described in Example 8 below.

Using any of the assays known in the art or described herein, the identified compounds from the screening methods (or an inhibitor employed in the therapeutic methods of the invention) can be additionally examined to confirm that they have no significant effect on tissue factor-mediated hemostasis activities (e.g., coagulation). For example, TF mediated coagulation activities can be measured by quantifying factor Xa generation in HaCaT cells by western blot, as demonstrated in the Examples below. These assays can be employed to examine test compounds used in the screening methods of the invention or inhibitor compounds used in the therapeutic methods of the invention. A compound does not interfere with or prevent activation of (i.e., having no significant effect on) a TF-mediated hemostasis (e.g., coagulation) if its presence does not lead to more than 5%, more than 10%, more than 15%, or more than 25% reduction in the hemostasis activity (e.g., coagulation activity as measured by the Xa generation assay under the conditions described herein) relative to that in the absence of the compound. In some embodiments, potential blocking activity of a compound on coagulation can be examined by assaying effect of the compound on the binding to tissue factor by an antibody which is known to block tissue factor mediated coagulation. One such antibody is the monoclonal antibody 5G9 produced by the hybridoma with ATCC access number HB9382. Inhibitory activities of this antibody on coagulation and relevant assays are disclosed in great detail in U.S. Pat. No. 5,223,427. A lack of significant effect of a compound on MAb 5G5 binding to tissue factor (e.g., a reduction of at least 20%, 30%, 40%, 50%, 75% or more) indicates that the compound is likely not to block tissue factor mediated coagulation.

Tissue factor signaling can also be detected and/or quantified using any of a number of well recognized immunological binding assays (see, e.g., U.S. Pat. Nos. 4,366,241; 4,376,110; 4,517,288; and 4,837,168). Antibodies useful in immunologic binding assays can act as an inhibitor of tissue factor signaling without interfering with hemostasis in a mammalian subject. The immunological binding assays utilize antibodies in the diagnosis or treatment of disease dependent upon altered tissue factor/factor VIIa signaling in a mammalian subject. For example, MAb 10H10 is an antibody that acts as an inhibitor of tissue factor signaling without interfering with hemostasis in the mammalian subject and is useful in immunologic binding assays as an embodiment of the invention. For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case tissue factor or antigenic subsequence thereof). The antibody (e.g., anti-tissue factor) can be produced by any of a number of means well known to those of skill in the art and as described above.

Immunoassays also often use a labeling agent to specifically bind to and label the complex formed by the antibody and antigen. The labeling agent can itself be one of the moieties comprising the antibody/antigen complex. Thus, the labeling agent can be a labeled tissue factor. Alternatively, the labeling agent can be a third moiety, such as a secondary antibody, that specifically binds to the antibody/tissue factor complex (a secondary antibody is typically specific to antibodies of the species from which the first antibody is derived). Other proteins capable of specifically binding immunoglobulin constant regions, such as protein A or protein G can also be used as the label agent. These proteins exhibit a strong non-immunogenic reactivity with immunoglobulin constant regions from a variety of species (see, e.g., Kronval et al., J. Immunol. 111: 1401-1406, 1973; Akerstrom et al., J. Immunol. 135: 2589-2542, 1985). The labeling agent can be modified with a detectable moiety, such as biotin, to which another molecule can specifically bind, such as streptavidin. A variety of detectable moieties are well known to those skilled in the art.

Throughout the assays, incubation and/or washing steps can be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-competitive assay formats: Immunoassays for detecting tissue factor signaling in samples can be either competitive or noncompetitive. Noncompetitive immunoassays are assays in which the amount of antigen is directly measured. In one preferred “sandwich” assay, for example, the anti-tissue factor antibodies can be bound directly to a solid substrate on which they are immobilized. These immobilized antibodies then capture tissue factor present in the test sample. Tissue factor thus immobilized are then bound by a labeling agent, such as a second antibody to tissue factor bearing a label. Alternatively, the second antibody can lack a label, but it can, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive assay formats: In competitive assays, the amount of tissue factor signaling present in the sample is measured indirectly by measuring the amount of a known, added (exogenous) tissue factor displaced (competed away) from an anti-tissue factor antibody by the unknown tissue factor present in a sample. In one competitive assay, a known amount of tissue factor is added to a sample and the sample is then contacted with an antibody that specifically binds to tissue factor. The amount of exogenous tissue factor bound to the antibody is inversely proportional to the concentration of tissue factor present in the sample. In a particularly preferred embodiment, the antibody is immobilized on a solid substrate. The amount of tissue factor bound to the antibody can be determined either by measuring the amount of tissue factor/antibody complex, or alternatively by measuring the amount of remaining uncomplexed protein. The amount of tissue factor can be detected by providing a labeled tissue factor molecule.

A hapten inhibition assay is another preferred competitive assay. In this assay the known tissue factor is immobilized on a solid substrate. A known amount of anti-tissue factor antibody is added to the sample, and the sample is then contacted with the immobilized tissue factor. The amount of anti-tissue factor antibody bound to the known immobilized tissue factor is inversely proportional to the amount of tissue factor present in the sample. Again, the amount of immobilized antibody can be detected by detecting either the immobilized fraction of antibody or the fraction of the antibody that remains in solution. Detection can be direct where the antibody is labeled or indirect by the subsequent addition of a labeled moiety that specifically binds to the antibody as described above.

Cross-reactivity determinations: Immunoassays in the competitive binding format can also be used for crossreactivity determinations. For example, tissue factor can be immobilized to a solid support. Proteins (e.g., tissue factor and homologs) are added to the assay that compete for binding of the antisera to the immobilized antigen. The ability of the added proteins to compete for binding of the antisera to the immobilized protein is compared to the ability of tissue factor to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of tissue factor, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of tissue factor that is required to inhibit 50% of binding, then the second protein is said to specifically bind to the polyclonal antibodies generated to tissue factor immunogen.

Other assay formats: Western blot (immunoblot) analysis is used to detect and quantify the presence of tissue factor in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind tissue factor. The anti-tissue factor antibody specifically binds to tissue factor on the solid support. These antibodies can be directly labeled or alternatively can be subsequently detected using labeled antibodies (e.g., labeled sheep anti-mouse antibodies) that specifically bind to the anti-tissue factor antibody.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5: 34-41, 1986).

Reduction of non-specific binding: One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels: The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), chemiluminescent labels, and colorimetric labels such as colloidal gold or colored glass or plastic beads (e.g., polystyrene, polypropylene, latex, etc.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize tissue factor, or secondary antibodies that recognize anti-tissue factor antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### Small Molecule Chemical Composition

“Small molecule” or “small chemical entity” includes any chemical or other moiety that can act to affect biological processes, wherein the small chemical entity can act as an inhibitor of tissue factor signaling without interfering with hemostasis in the mammalian subject, useful in the treatment or diagnosis of disease in a mammalian subject. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da. In the present method for treating disease dependent upon tissue factor/factor VIIa signaling in a mammalian subject, the small molecule organic compound, peptidomimetic, or antibody mimetics can be a mimetic of the antibody inhibitor, MAb 10H10.

Small molecules include without limitation organic compounds, peptidomimetics, antibody mimetics, and conjugates thereof. As used herein, the term “organic compound” or “small chemical entity” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocylcic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., Curr Pharm Des 6(10): 991-1007, 2000, Bioanalytical applications of accelerator mass spectrometry for pharmaceutical research; and Enjalbal et al., Mass Spectrom Rev 19(3): 139-61, 2000, Mass spectrometry in combinatorial chemistry.)

Preferred small molecules or small chemical entities are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

### High Throughput Assays for Modulators of Tissue Factor Signaling

As described above, the invention provides methods of identifying modulators, e.g., inhibitors or activators, of tissue factor signaling wherein the inhibitor does not interfere with hemostasis (e.g., in mammalian subjects). The test compounds to be employed in these methods can be any small organic molecule, or a biological entity, such as a protein, e.g. an antibody or peptide, a sugar, small chemical molecule, a nucleic acid, e.g., an antisense oligonucleotide, RNAi, or a ribozyme, or a lipid. Alternatively, modulators can be genetically altered versions of tissue factor. Typically, test compounds will be small organic molecules, peptides, antibodies, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int, J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Ramin, Wolburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Candidate compounds are useful as part of a strategy to identify drugs for treatment of a neoplastic disease or inflammatory disease wherein the compound inhibits tissue factor signaling and does not increase the risk of bleeding. A test compound that binds to signaling tissue factor is considered a candidate compound.

Screening assays for identifying candidate or test compounds that bind to tissue factor, or modulate the activity of tissue factor proteins or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some embodiments, the test compounds are activating variants of tissue factor.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or onphage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to inhibit the signaling activity of tissue factor or a biologically active portion thereof can be determined, e.g., by monitoring inhibition of tissue factor signaling in the absence of coagulation activity in the presence of the test compound. Modulating the activity of tissue factor or a biologically active portion thereof can be determined by measuring tissue factor signaling in the absence of coagulation activity. The ability of the test compound to modulate the tissue factor signaling, or a biologically active portion thereof, can also be determined by monitoring the ability of tissue factor to bind to protein disulfide isomerase. The binding assays can be cell-based or cell-free.

The ability of a compound to inhibit tissue factor signaling for treatment of a neoplastic disease or inflammatory disease without increasing the risk of bleeding can be determined by one of the methods described herein or known in the art for determining direct binding. In one embodiment, the ability of a compound to inhibit tissue factor signaling without increasing the risk of bleeding can be determined by monitoring tissue factor signaling in keartinocytes or endothelial cells. Detection of the tissue factor signaling can include detection of the expression of a recombinant tissue factor that also encodes a detectable marker such as a FLAG sequence or a luciferase. This assay can be in addition to an assay of direct binding. In general, such assays are used to determine the ability of a test compound to inhibit tissue factor signaling.

In general, the ability of a test compound to bind to tissue factor, interfere with tissue factor signaling is compared to a control in which the binding is determined in the absence of the test compound. In some cases, a predetermined reference value is used. Such reference values can be determined relative to controls, in which case a test sample that is different from the reference would indicate that the compound binds to the molecule of interest (e.g., tissue factor) or modulates tissue factor dependent PAR2 signaling in the presence of protein disulfide isomerase. A reference value can also reflect the amount of binding observed with a standard (e.g., the affinity of antibody for signaling tissue factor). In this case, a test compound that is similar to (e.g., equal to or less than) the reference would indicate that compound is a candidate compound (e.g., binds to signaling tissue factor to a degree equal to or greater than a reference antibody).

This invention further pertains to novel agents identified by the above-described screening assays and uses thereof for treatments as described herein.

In one embodiment the invention provides soluble assays using tissue factor, or a cell or tissue expressing tissue factor, either naturally occurring or recombinant. In another embodiment, the invention provides solid phase based in vitro assays in a high throughput format, where tissue factor, tissue factor in an appropriately modified conformation to mimic cellular signaling pools or its ligand is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for tissue factor protein in vitro, or for cell-based or membrane-based assays comprising tissue factor gene product or tissue factor protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as toll-like receptors, transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Bispecific Compounds as Modulators of Tissue Factor Signaling

In one aspect, a method for identifying candidate or test bispecific compounds is provided which reduce the concentration of an agent in the serum and/or circulation of a non-human animal. Compounds selected or optimized using the instant methods can be used to treat subjects that would benefit from administration of such a compound, e.g., human subjects.

Candidate compounds that can be tested in an embodiment of the methods of the present invention are bispecific compounds. As used herein, the term “bispecific compound” includes compounds having two different binding specificities. Exemplary bispecific compounds include, e.g., bispecific antibodies, heteropolymers, and antigen-based heteropolymers.

Bispecific molecules that can be tested in an embodiment of the invention preferably include a binding moiety that is specific for tissue factor, protein disulfide isomerase, or PAR2, preferably human tissue factor, protein disulfide isomerase, or PAR, crosslinked to a second binding moiety specific for a targeted agent (e.g. a distinct antibody or an antigen). Examples of binding moieties specific for tissue factor include, but are not limited to, tissue factor ligands, e.g., in preferred embodiments, antibodies to tissue factor signaling. The antibody can be an inhibitor of tissue factor signaling in a mammalian subject, wherein the inhibitor does not interfere with hemostasis in the mammalian subject.

In another embodiment, novel tissue factor binding molecules can be identified based on their ability to bind to tissue factor and inhibit tissue factor signaling. For example, libraries of compounds or small molecules can be tested cell-free binding assay. Any number of test compounds, e.g., peptidomimetics, small molecules or other drugs can be used for testing and can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12:145, 1997).

In many drug screening programs which test libraries of modulating agents and natural extracts, high throughput assays are desirable in order to maximize the number of modulating agents surveyed in a given period of time. Assays which are performed in cell-free systems, such as can be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as can be manifest in an alteration of binding affinity with upstream or downstream elements.

In another embodiment, phage display techniques known in the art can be used to identify novel tissue factor binding molecules. In one embodiment, the invention provides assays for screening candidate or test compounds which bind to tissue factor or biologically active portion thereof. Cell-based assays for identifying molecules that bind to tissue factor can be used to identify additional agents for use in bispecific compounds of the invention. For example, cells expressing tissue factor can be used in a screening assay. For example, compounds which produce a statistically significant change in binding to tissue factor can be identified.

In one embodiment, the assay is a cell-free assay in which a tissue factor binding molecule is identified based on its ability to bind to tissue factor protein in vitro. The tissue factor protein binding molecule can be provided and the ability of the protein to bind signaling tissue factor protein can be tested using art recognized methods for determining direct binding. Determining the ability of the protein to bind to a target molecule can be accomplished, e.g., using a technology such as real-time Biomolecular Interaction Analysis (BIA). Sjolander et al., Anal. Chem. 63: 2338-2345, 1991, and Szabo et al., Curr. Opin. Struct. Biol. 5: 699-705, 1995. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

The cell-free assays of the present invention are amenable to use of both soluble and/or membrane-bound forms of proteins. In the case of cell-free assays in which a membrane-bound form a protein is used it can be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPS O), or N-dodecyl=N,N-dimethyl-3-ammonio-1-propane sulfonate.

Suitable assays are known in the art that allow for the detection of protein-protein interactions (e.g., immunoprecipitations, two-hybrid assays and the like). By performing such assays in the presence and absence of test compounds, these assays can be used to identify compounds that modulate (e.g., inhibit or enhance) the interaction of a protein of the invention with a target molecule(s).

Determining the ability of the protein to bind to or interact with a target molecule can be accomplished, e.g., by direct binding. In a direct binding assay, the protein could be coupled with a radioisotope or enzymatic label such that binding of the protein to a target molecule can be determined by detecting the labeled protein in a complex. For example, proteins can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

Typically, it will be desirable to immobilize either a protein of the invention or its binding protein to facilitate separation of complexes from uncomplexed forms of one or both of the proteins, as well as to accommodate automation of the assay. Binding to an upstream or downstream binding element, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase (GST)/tissue factor fusion proteins can be adsorbed onto glutathione sepharose beads (Sigma Chemical, St. Louis, Mo.) or glutathione derivatized microtitre plates, which are then combined with the cell lysates, e.g. ³⁵S-labeled, and the test modulating agent, and the mixture incubated under conditions conducive to complex formation, e.g., at physiological conditions for salt and pH, though slightly more stringent conditions can be used. Following incubation, the beads are washed to remove any unbound label, and the matrix immobilized and radiolabel determined directly (e.g. beads placed in scintillant), or in the supernatant after the complexes are subsequently dissociated. Alternatively, the complexes can be dissociated from the matrix, separated by SDS-PAGE, and the level of tissue factor-binding protein found in the bead fraction quantitated from the gel using standard electrophoretic techniques.

Other techniques for immobilizing proteins on matrices are also available for use in the subject assay. For instance, biotinylated molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical).

It is also within the scope of this invention to determine the ability of a compound to modulate the interaction between tissue factor and protein disulfide isomerase or tissue factor and PAR, without the labeling of any of the interactants. For example, a microphysiometer can be used to detect the interaction of a protein of the invention with its target molecule without the labeling of either the protein or the target molecule. McConnell et al., Science 257: 1906-1912, 1992. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

Antigen-based heteropolymers that can be tested in the present invention preferentially include a binding moiety that is specific for tissue factor, preferably human tissue factor, crosslinked to an antigen that is recognized by an autoantibody. Examples of antigens recognized by autoantibodies include, but are not limited to, any one of the following: factor VIII (antibodies associated with treatment of hemophilia by replacement recombinant factor VIII); the muscle acetylcholine receptor (the antibodies are associated with the disease myasthenia gravis); cardiolipin (associated with the disease lupus); platelet associated proteins (associated with the disease idiopathic thrombocytopenic purpura); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is well-characterized and is associated with disease pathogenesis.

Exemplary heteropolymers and antigen-based heteropolymers for testing in the instant invention and methods of making them are known in the art. For example, exemplary heteropolymers are taught in WO 03007971A1; U.S. 20020103343A1; U.S. Pat. No. 5,879,679; U.S. Pat. No. 5,487,890; U.S. Pat. No. 5,470,570; WO 9522977A1; WO/02075275A3, WO/0246208A2 or A3, WO/0180883A1, WO/0145669A1, WO 9205801A1, Lindorfer et al., J. Immunol. Methods. 248: 125, 2001; Hahn et al., J. Immunol. 166: 1057, 2001; Nardin et al., J. Immunol. Methods. 211: 21, 1998; Kuhn et al., J. Immunol. 160: 5088, 1998; Taylor et al., Cancer Immunol. Immunother. 45: 152, 1997; Taylor et al., J. Immunol. 159: 4035, 1997; and Taylor et al., J. Immunol. 148: 2462, 1992. In addition, variant forms of these heteropolymers can be made. For example, in one embodiment, forms of bispecific molecules made using different linking chemistries can be used. Exemplary reagents that can be used to cross-link the components of a bispecific molecule include: polyethelyene glycol, SATA, SMCC, as well others known in the art, and available, e.g., from Pierce Biotechnology. Exemplary forms of bispecific molecules that can be tested are described in U.S. Ser. No. 60/411,731, filed on Sep. 16, 2002, the contents of which are incorporated herein by reference.

In another embodiment, different multimeric forms of bispecific molecules can be made (e.g., dimer, trimer, tetramer, pentamer, or higher multimer forms). In another embodiment, purified forms of bispecific molecules can be tested, e.g., as described in U.S. Ser. No. 60/380,211, filed on May 13, 2002, the contents of which are incorporated herein by reference.

In another embodiment, when one of the binding moieties of the heteropolymer is an antibody, antibodies of different isotypes (e.g., IgA, IgD, IgE, IgG1, IgG₂ (e.g., IgG₂a), IgG₃, IgG₄, or IgM) can be used. In another embodiment, portions of an antibody molecule (e.g., Fab fragments) can be used for one of the binding moieties. In a preferred embodiment at least one of the binding moieties is an antibody comprising an Fc domain. In one embodiment, the antibody is a mouse antibody.

In another embodiment, the effect of modifications to antibodies can be tested, e.g., the effect of deimmunization of the antibody, e.g., as described in U.S. Ser. No. 60/458,869, filed on Mar. 28, 2003 can be tested.

In methods provided in the present invention, the concentration of an agent, e.g. pathogenic agent, in the serum, circulation and/or tissue of the non-human animal can be reduced by at least e.g. about 20%, about 30%, about 40%, about 50%, about 60%, about 70%, about 80%, about 90% or about 100%.

In another embodiment, the concentration of an agent in the serum, circulation and/or tissue of a subject can be measured indirectly. For example, pathology resulting from the presence of the agent in the serum and/or circulation can be measured, e.g., by examining tissue samples from the animal. Another indirect measurement of the concentration of an agent in the serum, circulation and/or tissue of the non-human animal is measurement of the ability of the agent to cause infection in the non-human animal. For example, the effect of the bispecific compound on clinical signs and symptoms of infection can be measured. The ability of the bispecific compound to inhibit the spread of infection, e.g., from one organ system to another or from one individual to another can also be tested.

In another embodiment the ability of the bispecific compound to bind to cells bearing tissue factor in the non-human animal is measured. For example, in one embodiment, determining the ability of the bispecific compound to bind to a tissue factor target molecule can also be accomplished using a technology such as real-time Biomolecular Interaction Analysis (BIA) (Sjolander et al., Anal. Chem. 63: 2338-2345, 1991 and Szabo et al., Curr. Opin. Struct. Biol. 5: 699-705, 1995). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, the destruction of the agent by cells in the non-human animal (e.g., killing by macrophage) is measured.

Compounds that reduce the concentration of the agent in the serum and/or circulation of the non-human animal (as compared with concentrations observed in non-human animals that do not receive the bispecific compound) can be selected.

Compounds for testing in the subject assays can be selected from among a plurality of compounds tested. In another embodiment, bispecific compounds for testing in the instant assays may have already been identified as being capable of binding tissue factor, e.g., in an in vitro assay and can be further evaluated or optimized using the instant assays. In such cases, the ability of a bispecific compound to reduce the concentration of an agent in the serum and/or circulation can be compared to another bispecific compound or a non-optimized version of the same compound to determine its ability reduce the concentration of the agent in the serum and/or circulation.

In preferred embodiments, the bispecific compounds of the instant invention are administered at concentrations in the range of approximately 1 μg compound/kg of body weight to approximately 100 μg compound/kg of body weight. As defined herein, a therapeutically effective amount of a bispecific compound (i.e., an effective dosage) ranges from about 0.01 to 5000 μg/kg body weight, preferably about 0.1 to 500 μg/kg body weight, more preferably about 2 to 80 μg/kg body weight, and even more preferably about 5 to 70 μg/kg, 10 to 60 μg/kg, 20 to 50 μg/kg, 24 to 41 μg/kg, 25 to 40 μg/kg, 26 to 39 μg/kg, 27 to 38 μg/kg, 28 to 37 μg/kg, 29 to 36 μg/kg, 30 to 35 μg/kg, 31 to 34 μg/kg or 32 to 33 μg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, the animal is treated with bispecific compound in the range of between about 1 to 500 μg/kg body weight following intravenous (iv) injection of an agent. It will also be appreciated that the effective dosage of a bispecific compound used for treatment can increase or decrease over the course of a particular treatment. Changes in dosage may result and become apparent from the results of diagnostic assays as described herein.

The route of administration of test compounds and/or agents can be intravenous (iv) injection into the circulation of the animal. Other administration routes include, but are not limited to, topical, parenteral, subcutaneous, or by inhalation. The term “parenteral” includes injection, e.g. by subcutaneous, intravenous, or intramuscular routes, also including localized administration, e.g., at a site of disease or injury. Sustained release of compounds from implants is also known in the art. One skilled in the pertinent art will recognize that suitable dosages will vary, depending upon such factors as the nature of the disorder to be treated, the patient's body weight, age, and general condition, and the route of administration. Preliminary doses can be determined according to animal tests, and the scaling of dosages for human administration are performed according to art-accepted practices.

The candidate compounds and agents can be administered over a range of doses to the animal. When the agent is also administered to the animal, the candidate compound can be administered either before, at the same time, or after, administration of the agent.

Tissue factor expressing transgenic animals, e.g mice, of the present invention can be used to screen or evaluate candidate compounds useful for treating disorders or diseases in humans that are associated with the presence of unwanted agents in the serum and/or circulation of a subject, such as autoantibodies, infectious agents, or toxins.

Exemplary targeted agents that can be bound by the bispecific compounds of the present invention include blood-borne agents, including, but not limited to, any of the following: viruses, tumor cells, inflammatory cells, polynucleotides, antibodies, e.g., autoantibodies associated with an autoimmune disorder.

In one embodiment, in performing an assay of the invention, the agent is administered to the transgenic animal, e.g., prior to, simultaneously with, or after administration of a bispecific compound.

The bispecific compounds of the present invention, or any portion thereof, can be modified to enhance their half life. Peptide analogs are commonly used in the pharmaceutical industry as non-peptide drugs with properties analogous to those of the template peptide. These types of non-peptide compounds are termed “peptide mimetics” or “peptidomimetics” (Fauchere, Adv. Drug Res. 15: 29, 1986; Veber et al., TINS p. 392, 1985; and Evans et al., J. Med. Chem. 30: 1229, 1987, which are incorporated herein by reference) and are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), such as an antigen polypeptide, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins Weinstein, B., ed., Marcel Dekker, New York, p. 267, 1983; Spatola, A. F., Vega Data, Vol. 1, Issue 3, “Peptide Backbone Modifications,” 1983; Morley, Trends. Pharm. Sci. pp. 463-468, 1980; Hudson et al., Int. J. Pept. Prot. Res. 14: 177-185, 1979 (—CH₂NH—, CH₂CH₂—); Spatola et al., Life. Sci. 38: 1243-1249, 1986 (—CH₂—S); Hann, J. Chem. Soc. Perkin. Trans. 1: 307-314, 1982 (—CH—CH—, cis and trans); Almquist et al., J. Med. Chem. 23: 1392-1398, 1980 (—COCH₂—); Jennings-White et al., Tetrahedron Lett. 23: 2533, 1982 (—COCH₂—); Szelke et al., European Patent Application No. EP 45665 CA: 97: 39405, 1982 (—CH(OH)CH₂—); Holladay et al., Tetrahedron. Lett. 24: 4401-4404, 1983 (—C(OH)CH₂—); and Hruby, Life Sci. 31: 189-199, 1982 (—CH₂—S—); each of which is incorporated herein by reference. A particularly preferred non-peptide linkage is —CH₂NH—. Such peptide mimetics can have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) can be used to generate more stable peptides. In addition, constrained peptides can be generated by methods known in the art (Rizo et al., Annu. Rev. Biochem. 61: 387, 1992, incorporated herein by reference); for example, by adding internal cysteine residues capable of forming intramolecular disulfide bridges which cyclize the peptide.

Such modified polypeptides can be produced in prokaryotic or eukaryotic host cells. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual, 2nd Ed., Cold Spring Harbor, N.Y., 1989; Berger et al., Methods in Enzymology, Volume 152, Guide to Molecular Cloning Techniques, 1987, Academic Press, Inc., San Diego, Calif.; Merrifield, J. Am. Chem. Soc. 91: 501, 1969; Chaiken, CRC Crit. Rev. Biochem. 11: 255, 1981; Kaiser et al., Science 243: 187, 1989; Merrifield, Science 232: 342, 1986; Kent, Annu. Rev. Biochem. 57: 957, 1988; and Offord, Semisynthetic Proteins, Wiley Publishing, 1980, which are incorporated herein by reference).

Polypeptides can be produced, typically by direct chemical synthesis, and used as a binding moiety of a heteropolymer. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal modifications such as amidation, as well as other terminal modifications, including cyclization, can be incorporated into various embodiments of the test compounds. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

### Detectable Label

The particular label or detectable group used in the assay can be detectable by spectroscopic, photochemical, biochemical, immunochemical, electrical, optical or chemical means. The particular type of label is not a critical aspect of the invention, so long as it does not significantly interfere with the specific binding of an antibody to the signaling tissue factor, e.g., Mab 10H10, used in the assay. The detectable group can be any material having a detectable physical or chemical property. Such detectable labels have been well-developed in the field of assays or immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹¹²In, ⁹⁹mTc), other imaging agents such as microbubbles (for ultrasound imaging), ¹⁸F, ¹¹C, ¹⁵O, (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g. polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazinediones, e.g., luminol. For a review of various labeling or signal producing systems which can be used, see, U.S. Pat. No. 4,391,904, incorporated herein by reference in its entirety and for all purposes.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple calorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Frequently, the cellular marker and antibodies to the cellular marker will be labeled by joining, either covalently or non-covalently, a substance which provides for a detectable signal.

### Kits

Also within the scope of the invention are kits comprising the compositions (e.g., monoclonal antibodies, human sequence antibodies, human antibodies, multispecific and bispecific molecules, small chemical molecules, nucleic acid compositions, e.g., antisense oligonucleotides, double stranded RNA oligonucleotides (RNAi, shRNA, siRNA), or DNA oligonucleotides or vectors containing nucleotide sequences encoding for the transcription of shRNA molecules of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

### Pharmaceutical Compositions

Therapeutic compositions (e.g., monoclonal antibodies, human sequence antibodies, human antibodies, multispecific and bispecific molecules, small chemical molecules, nucleic acid compositions, e.g., antisense oligonucleotides, double stranded RNA oligonucleotides (RNAi, shRNA, siRNA), or DNA oligonucleotides or vectors containing nucleotide sequences encoding for the transcription of siRNA molecules are useful in the present compositions and methods to be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, cancer or metastatic cancer.

Pharmaceutically acceptable carriers are determined in part by the particular composition being administered, as well as by the particular method used to administer the composition. Accordingly, there is a wide variety of suitable formulations of pharmaceutical compositions for administering the therapeutic antibody in combination with tumor cell targeting or small molecule or ligand compositions (see, e.g., Remington's Pharmaceutical Sciences, Mack Publishing Co., Easton, Pa. 18^{th} ed., 1990, incorporated herein by reference). The pharmaceutical compositions generally comprise a differentially expressed protein, agonist or antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

### Treatment Regimes

The invention provides pharmaceutical compositions comprising one or a combination of compositions (e.g., monoclonal antibodies, human sequence antibodies, human antibodies, multispecific and bispecific molecules, small molecules, ligand mimetics, derivatives and analogs thereof nucleic acid compositions, e.g., antisense oligonucleotides, double stranded RNA oligonucleotides (RNAi, shRNA, siRNA), or DNA oligonucleotides or vectors containing nucleotide sequences encoding for the transcription of shRNA molecules that specifically binds to signaling tissue factor in a neoplastic tumor cell or inflammatory cell, are formulated together with a pharmaceutically acceptable carrier. Some compositions include a combination of multiple (e.g., two or more) antibody or small molecule therapeutics.

In prophylactic applications, pharmaceutical compositions or medicaments are administered to a patient susceptible to, or otherwise at risk of a disease or condition (i.e., an immune disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

### Effective Dosages

Effective doses of the pharmaceutical compositions (e.g., monoclonal antibodies, human sequence antibodies, human antibodies, multispecific and bispecific molecules, small chemical molecules, nucleic acid compositions, e.g., antisense oligonucleotides, double stranded RNA oligonucleotides (RNAi, shRNA, siRNA), or DNA oligonucleotides or vectors containing nucleotide sequences encoding for the transcription of shRNA molecules) that inhibit tissue factor signaling, or other inhibitors of tissue factor, e.g., small molecule inhibitors, for the treatment of neoplastic disease or inflammatory disease, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with an therapeutic antibody or small molecule composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more therapeutic antibody or small molecule compositions with different binding target specificities are administered simultaneously, in which case the dosage of each therapeutic antibody or small molecule composition administered falls within the ranges indicated. A therapeutic antibody or small molecule composition is usually administered on multiple occasions. Intervals between single dosages can be weekly, monthly or yearly. Intervals can also be irregular as indicated by measuring blood levels of therapeutic antibody or small molecule composition in the patient. In some methods, dosage is adjusted to achieve a plasma antibody or small molecule composition concentration of 1-1000 μg/ml and in some methods 25-300 μg/ml. Alternatively, an antibody or small molecule composition can be administered as a sustained release formulation, in which case less frequent administration is required. Dosage and frequency vary depending on the half-life of the therapeutic antibody or small molecule composition in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for therapeutic antibody or small molecule composition range from about 10 ng to 1 g, 100 ng to 100 mg, 1 μg to 10 mg, or 30-300 μg per patient.

### Routes of Administration

Therapeutic compositions (e.g., monoclonal antibodies, human sequence antibodies, human antibodies, multispecific and bispecific molecules, small chemical molecules, nucleic acid compositions, e.g., antisense oligonucleotides, double stranded RNA oligonucleotides (RNAi, shRNA, siRNA), or DNA oligonucleotides or vectors containing nucleotide sequences encoding for the transcription of shRNA molecules, for the treatment of neoplastic disease, or inflammatory disease can be administered by parenteral, topical, intravenous, oral, subcutaneous, intraarterial, intracranial, intraperitoneal, intranasal or intramuscular means for prophylactic as inhalants for therapeutic antibody or small molecule preparations targeting neoplastic disease or inflammatory disease, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where a tumor is found, for example intracranial injection or convection enhanced delivery. Intramuscular injection or intravenous infusion are preferred for administration of an antibody or small molecule composition. In some methods, particular therapeutic antibody or small molecule composition are delivered directly into the cranium. In some methods, antibody or small molecule composition is administered as a sustained release composition or device, such as a Medipad™ device.

Agents of the invention can optionally be administered in combination with other agents that are at least partly effective in treating various diseases including various immune-related diseases. In the case of tumors in the brain, both primary and metastatic, therapeutic compositions can also be administered in conjunction with other agents that increase passage of the agents of the invention across the blood-brain barrier (BBB). For example, intranasal delivery of therapeutic antibody or small molecule composition can include cell membrane penetration enhancers.

### Formulation

Compositions (e.g., monoclonal antibodies, human sequence antibodies, human antibodies, multispecific and bispecific molecules, small chemical molecules, nucleic acid compositions, e.g., antisense oligonucleotides, double stranded RNA oligonucleotides (RNAi, shRNA, siRNA), or DNA oligonucleotides or vectors containing nucleotide sequences encoding for the transcription of shRNA molecules, for the treatment of neoplastic disease, or inflammatory disease, are often administered as pharmaceutical compositions comprising an active therapeutic agent, e.g., a chemotherapeutic agent or anti-inflammatory agent, and a variety of other pharmaceutically acceptable components. See Remington's Pharmaceutical Science (15^{th} ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, a therapeutic antibody or small molecule composition, can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Therapeutic antibody or small molecule composition can be administered in the form of a depot injection or implant preparation which can be formulated in such a manner as to permit a sustained release of the active ingredient. An exemplary composition comprises a therapeutic antibody or small molecule composition at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in fall compliance with all Good Manufacturing Practice (GMP) regulations of the U.S. Food and Drug Administration.

### Toxicity

Preferably, a therapeutically effective dose of compositions (e.g., monoclonal antibodies, human sequence antibodies, human antibodies, multispecific and bispecific molecules, small chemical molecule, nucleic acid compositions, e.g., antisense oligonucleotides, double stranded RNA oligonucleotides (RNAi, shRNA, siRNA), or DNA oligonucleotides or vectors containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1.

The following examples of specific embodiments for carrying out the present invention are offered for illustrative purposes only, and are not intended to limit the scope of the present invention in any way.

### EXEMPLARY EMBODIMENTS

As illustrated in the Examples below, the present inventors discovered that TF-VIIa mediated coagulation and cell signaling involve distinct cellular pools of TF. It was found that the surface accessible, extracellular Cys¹⁸⁶-Cys²⁰⁹ disulfide bond of TF is required for coagulation activation as well as coagulation initiation phase signaling by Xa in the ternary TF-VIIa-Xa complex, but not for direct PAR2 cleavage by the binary TF-VIIa complex. Mutational breaking of this disulfide recapitulates the functional properties of the TF-VIIa signaling pool which has low affinity for VIIa on cells with constitutive TF expression.

In addition, it was observed that protein disulfide isomerase (PDI) disables coagulation by targeting this disulfide. TF coagulant activity is suppressed upon association of extracellular PDI with TF and that disulfide/thiol exchange pathways are required for TF-PAR2 complex formation and TF-VIIa signaling. There is a close correlation between TF-PDI association and TF-VIIa signaling in several cell types, including breast cancer cells. A unique monoclonal antibody (MAb-10H10) only recognizes the non-coagulant, cryptic conformation of TF. This antibody inhibits formation of the TF-PAR complex and TF-VIIa signaling, but does not prevent coagulation activation. Blockade of TF-VIIa signaling in these cells by MAb-10H10 is superior to blocking coagulation by MAb-5G9 to suppress tumor growth (e.g., breast tumor or melanoma), emphasizing the relevance of the TF-VIIa signaling pathway in vivo. Importantly, MAb-10H10 has minimal effects on coagulation activation, indicating that the inhibition of TF-VIIa signaling does not impair hemostasis.

It was additionally discovered that PDI suppresses TF coagulant activity in a nitric oxide (NO)-dependent pathway. Vascular protective NO synthesis is frequently perturbed in atherosclerosis, diabetes or inflammation and uncoupling of nitric oxide synthesis may shift cell surface TF activity to coagulation. NO-dependent inhibition of TF coagulant activity thus links the regulation of thrombogenicity in an unexpected way to oxidative stress in cardiovascular disease and inflammation.

### Example 1

### Specific Inhibition of Signaling TF

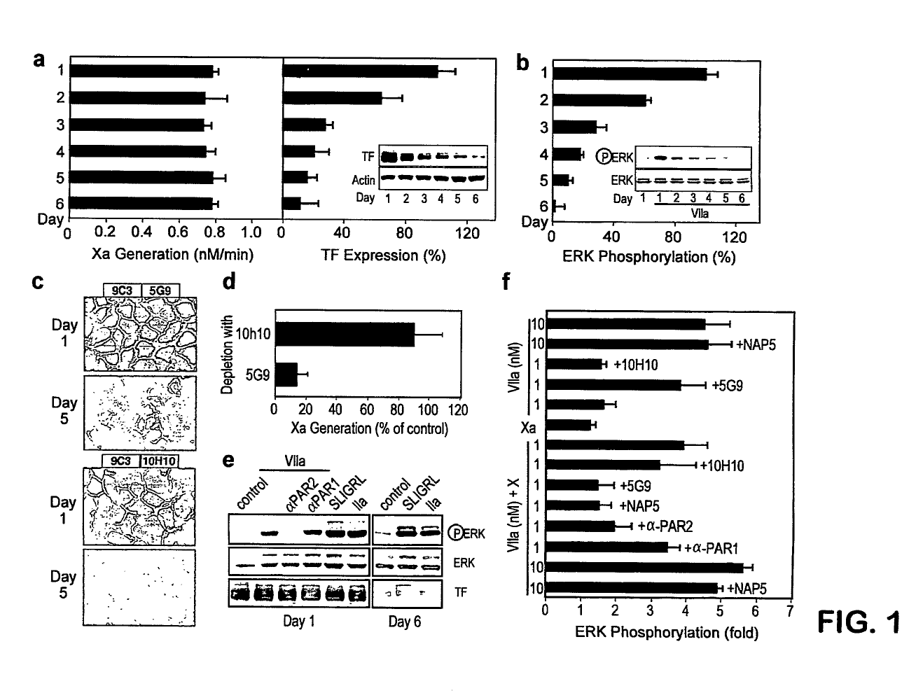

Cell surface-expressed TF binds VIIa with variable affinity and TF coagulation activation is typically saturated at lower VIIa concentrations relative to cell signaling. Le et al., J. Biol. Chem. 267: 15447-15454, 1992; Hjortoe et al., Blood 103: 3029-3037, 2004. It is unclear whether differential VIIa binding is inherent to the conformation of TF or due to other factors that are known to influence coagulant activity of TF, including the lipid environment, chaperone proteins, or calcium mobilization. Sevinslcy et al., J. Cell Biol. 133: 293-304, 1996; Carson et al., Blood 84: 526-534, 1994; Bhattacharjee et al., Arterioscler. Thromb. Vasc. Biol. 25: 1737-1743, 2005; Bach and Moldow, Blood 89: 3270-3276, 1997. We found that TF's coagulant activity remained unchanged despite progressive downregulation of TF expression levels to <5% in human keratinocytes during growth arrest, documented by constant actin levels (FIG. 1a). In contrast, TF-VIIa signaling was downregulated concordant with TF levels (FIG. 1b), although PAR responsiveness was not lost (FIG. 1e).

Staining of non-permeabilized cells confirmed reduced cell surface expression of TF (FIG. 1c), but unexpectedly we found epitope loss for MAb-10H10 that binds to the TF carboxyl-terminus without inhibiting VIIa association. Ruf et al., Biochem. J. 278: 729-733, 1991. Reactivity of MAb-5G9 which inhibits coagulation and substrate binding was preserved, indicating that MAb-10H10 lost reactivity with the residual pool of TF that drives coagulation. Huang et al., J. Mol. Biol. 275: 873-894, 1998. Immunodepletion experiments using purified, phospholipid reconstituted TF or cell lysates of TF expressing cells confirmed that only MAb 5G9, but not MAb 10H10 efficiently bound coagulant TF (FIG. 1d). Thus, antigenically distinct pools of TF appear to be responsible for coagulation versus TF-VIIa cell signaling through PAR2 (FIG. 1e).

Irrespectively of time of culture, coagulant TF had high affinity for VIIa, as rates of Xa generation were saturated to 99% at 1 nM VIIa. In the TF-VIIa-X coagulation initiation complex, the nascent product Xa rather than TF-VIIa activates PARs. Riewald and Ruf, Proc. Natl. Acad. Sci. USA 98: 7742-7747, 2001. Low (1 nM) VIIa concentrations produced appreciable signaling only in the presence of substrate X and signaling of TF-VIIa-Xa was blocked by the Xa inhibitor NAP5 (FIG. 1f). TF-VIIa signaling at 10 nM VIIa was not inhibited by NAP5 and NAP5 had only a minimal effect on signaling when substrate X was added together with 10 nM VIIa. This shows that the TF-VIIa signaling pool is not affected by the presence of substrate. Coagulation and Xa-dependent signaling of the coagulation initiation complex were inhibited by MAb-5G9, but not MAb-10H10. Importantly, MAb-10H10 with poor reactivity towards coagulant TF (FIGS. 1c,d) blocked TF-VIIa signaling. Thus, we identified a specific inhibitory antibody to signaling TF which does not interfere with coagulation initiation or ternary TF-VIIa-Xa complex signaling.

FIG. 1 shows specific inhibition of signaling TF. a Coagulant activity, TF expression and b TF-VIIa signaling in growth arrested HaCaT cells, mean±sd (n=3). Insets: actin or TF in cell lysates. c Cell surface TF detection with FITC-conjugated MAb-9C3 and Texas Red-conjugated MAb-5 G9 or 10H10. d Xa generation of phospholipid-reconstituted TF immunodepleted by immobilized MAbs-10H10 or 5G9 relative to control. e PAR2-dependence of HaCaT TF-VIIa signaling. f Different affinity for VIIa distinguishes TF that mediates TF-VIIa or Xa-dependent, ternary TF-VIIa-Xa complex signaling. MAb-10H10 specifically inhibits TF-VIIa signaling; mean±sd (n>3).

### Example 2

### Signaling TF is Regulated by Protein Disulfide Isomerase

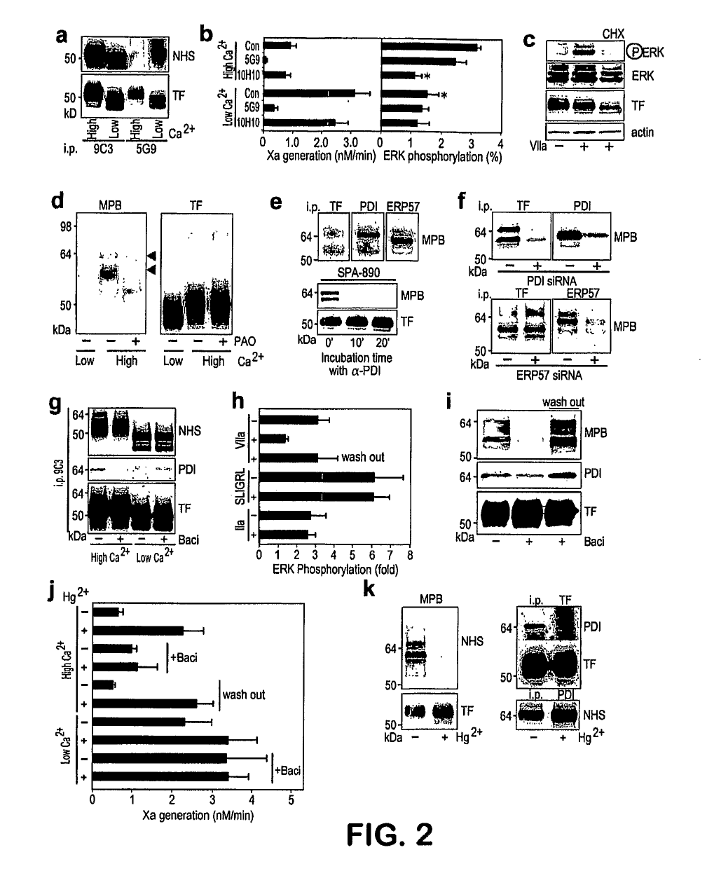

To further address the regulation of TF function in this model, cells were replated into Ca²⁺-depleted medium to prevent cell-cell contacts for 48 hours. Alternatively, medium was supplemented for the last 24 hours with 2 mM Ca²⁺ (high Ca²⁺) which induced distinct epithelial morphology without affecting levels of TF expression or VIIa binding. Equivalent and predominant cell surface expression of TF were confirmed by surface biotinylation and immunoprecipitation with MAb-5G9 which does not bind TF after biotinylation (FIG. 2a). However, TF showed marked functional differences under these two conditions (FIG. 2b) and coagulant activity was ˜3-4 fold higher in low Ca²⁺ cells. Although direct PAR2 activation with agonist peptide was comparable, TF-VIIa signaling was prominent only in high, but not low Ca²⁺ cells. Thus, the change to high Ca²⁺ appeared to induce signaling TF at the expense of coagulant TF. Consistently, blocking protein synthesis with cycloheximide (CHX) at the time of Ca²⁺ addition prevented TF-VIIa signaling concomitantly with blocking the appearance of higher glycosylated, cell surface TF (FIG. 2c).

Because protein disulfide isomerase (PDI) is induced during contact inhibition, we reasoned that TF's coagulant activity was suppressed by a PDI pathway that targets the solvent exposed TF Cys¹⁸⁶-Cys²⁰⁹ disulfide bond required for coagulation. Rehemtulla et al., J. Biol. Chem. 266: 10294-10299, 1991. Such cross strand disulfides are susceptible to reduction due to the strained bond geometry. Hogg, Trends Biochem. Sci. 28: 210-214, 2003; Wouters et al., BioEssays 26: 73-79, 2004. Cell surface PDI was found associated with TF, based on labelling with the membrane impermeable, thiol-reactive 3-(N-maleimido-propionyl)biocytin (MPB). MPB-labeled bands of ˜56 and 64 kDa coprecipitated with TF specifically from high Ca²⁺ cells (FIG. 2d), but blocking vicinal thiols with phenylarsine oxide (PAO) abolished labelling. A faint, variably labelled band at the appropriate molecular weight for TF remained, possibly indicating transient reduction of TF. The major MPB-labelled bands co-precipitating with TF aligned with immunoprecipitated PDI, but not the close homologue ERP57, and anti-PDI blocked MPB labelling (FIG. 2e). Knockdown of PDI or ERP57 with siRNA further validated that PDI co-precipitated with TF (FIG. 2f).

Communoprecipitation of NHS surface-biotinylated PDI at 64 kDa was confirmed by Westernblotting (FIG. 2g). Bacitracin, an inhibitor of PDI prevented the co-precipitation of PDI with TF. Mandel et al., Proc. Natl. Acad. Sci. U.S.A 90: 4112-4116, 1993. Bacitracin inhibited TF-VIIa signaling, but not direct PAR2 agonist or thrombin responses (FIG. 2h). Washout of bacitracin restored TF-VIIa signaling and MPB-labeling of surface PDI showed concomitant rapid re-association of PDI with TF within the time frames of these experiments (FIG. 2i). Thus, signaling TF on high Ca²⁺ cells is in a bacitracin-sensitive complex with PDI.

Although the presence of bacitracin slightly increased coagulant function of TF (FIG. 2j), activity on low versus high Ca²⁺ cells remained strikingly different. We assume that the partial effect of adding bacitracin on TF activity is due to inefficient dissociation of PDI relative to the labelling conditions (FIGS. 2g,i). A brief exposure to HgCl₂ increased TF coagulant activity substantially in high Ca²⁺ cells, indicating that TF is negatively regulated by reductive pathways. The increased coagulation due to oxidation is influencing TF, because HgCl₂ had little effect in low Ca²⁺ cells, did not increase coagulation in the presence of bacitracin and specifically disrupted the association of PDI with TF without displacing PDI from the cell surface (FIG. 2k). Furthermore, bacitracin was a reversible inhibitor of HgCl₂ activation (FIG. 2j), but did not block non-specific, toxic effects of HgCl₂ on cells. Thus, signaling TF lost coagulant activity by a redox mechanism. MAb-10H10, but not other antibody reactivity was diminished upon surface oxidation, further documenting a redox-sensitive conformation of signaling TF.

FIG. 2 shows signaling TF is regulated by PDI. a Similar TF cell surface expression upon high Ca²⁺ switch documented by NHS surface-biotinylated that prevents MAb-5G9 immunoprecipitation. b Low coagulant activity of TF in high Ca²⁺ cells is associated with MAb-10H10 inhibitable TF-VIIa signaling; * different from high Ca²⁺ control, p<0.01, t-test, mean±sd (n>4), c Cycloheximide (CHX) TF synthesis block prevents TF-VIIa signaling. d MPB labelling of proteins co-precipitating with TF is inhibited by 2 μM PAO. E Co-migration of MPB-labelled bands in TF and PDI immunoprecipitates. Anti-PDI SPA-890 blocks MPB labelling f PDI, but not ERP57 knockdown with siRNA prevents MPB-labelled bands in TF immunoprecipitates. g PDI-inhibitor bacitracin (3 mM) dissociates PDI from TF in MAb-9C3 immunoprecipitates of NHS surface-biotinylated cells. h Bacitracin reversibly blocks TF-VIIa signaling. Washout: cells preincubated for 10 minutes with bacitracin were washed prior to stimulation with 10 nM VIIa; * p<0.01 relative to control, t-test, mean±sd (n>4). i Wash-out of bacitracin promotes PDI-TF reassociation; labelling 10 minutes after wash. j Effect of bacitracin on TF coagulant activity. The cell surface was oxidized with 100 μM HgCl₂ for 2 minutes prior to functional assay. k Oxidation with HgCl₂ dissociates PDI from TF without displacing MPB- or NHS-biotinylated PDI from the cell surface.

### Example 3

### Mutational Breaking of the TF Disulfide Recapitulated Reduced Affinity for VIIa, the Hallmark of Signaling TF

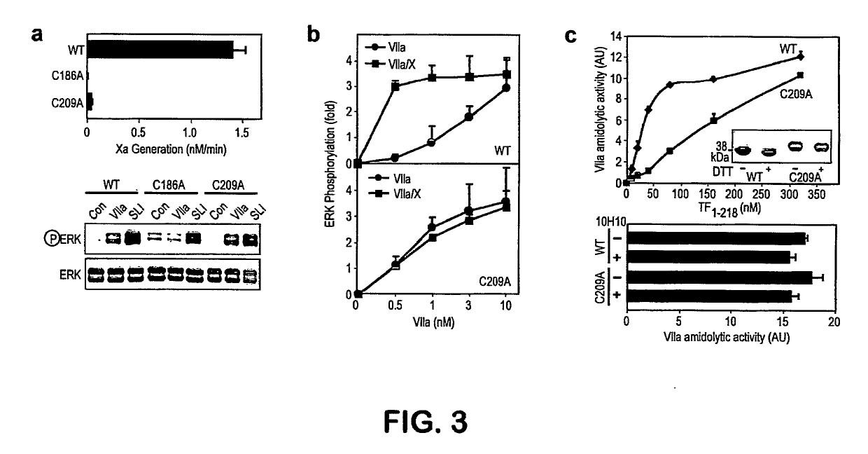

We tested whether a broken TF Cys¹⁸⁶-Cys²⁰⁹ disulfide recapitulated signaling properties of PDI-regulated TF. Individual alanine substitution mutants for the disulfide were introduced into TF-negative umbilical vein endothelial cells (HUVECs) by adenoviral transduction to achieve similar surface expression. Each mutant showed >95% diminished TF-VIIa mediated Xa generation, but TF-VIIa signaling was preserved in PAR2-agonist responsive cells that expressed C209A, but not C186A TF (FIG. 3a). VIIa binding to C209A TF by indirect immunofluorescence was comparable to wild-type TF. TF-VIIa signaling in wild-type TF expressing cells required >1 nM VIIa and ternary TF coagulation initiation complex signaling occurred at <1 nM VIIa (FIG. 3b). In cells transduced with C209A TF, TF-VIIa signaling required somewhat lower concentrations of VIIa relative to wild-type TF expressing cells. Importantly, C209A TF-mediated signaling did not change when substrate X was added with VIIa. Thus, formation of the Cys¹⁸⁶-Cys²⁰⁹ disulfide was required to generate high affinity TF that not only drives coagulation, but also ternary TF-VIIa-Xa complex signaling.

We confirmed that reduced C209A TF allosterically induces the catalytic activity of VIIa which is necessary for proteolytic cell signaling. Recombinant soluble monomeric C209A TF stimulated VIIa activity maximally at saturation. However, reduced C209A TF had lower affinity to enhance VIIa's catalytic activity relative to soluble, oxidized wild-type TF (FIG. 3C). Thus, mutational breaking of the TF disulfide recapitulated reduced affinity for VIIa, the hallmark of signaling TF. MAb-10H10 did not block catalytic activity of C209A TF-VIIa, excluding that the antibody inhibits VIIa binding specifically to signaling TF. MAb 10H10 thus likely prevents TF-VIIa mediated PAR cleavage by steric hindrance.

FIG. 3 shows signaling of reduced TF. a Mutant C209A TF-VIIa signals, but loses coagulant activity in HUVECs expressing similar levels of wild-type, C186A or C209A TF with PAR. b Dose response of VIIa signaling with and without 100 nM X in HUVECs expressing C209A or wild-type TF, mean±sd (n>4). c Recombinant soluble C209A TF activates VIIa (40 nM) with reduced affinity versus wild-type TF; mean±sd (n=3). Inset gel of homogenous preparations of monomeric soluble TF; the expression tag was not cleaved off from C209A TF yielding higher molecular weight. Lower panel: MAb-10H10 has no effect on TF-VIIa amidolytic activity.

### Example 4

### Role of Coagulant TF and Signaling TF in Tumor Progression

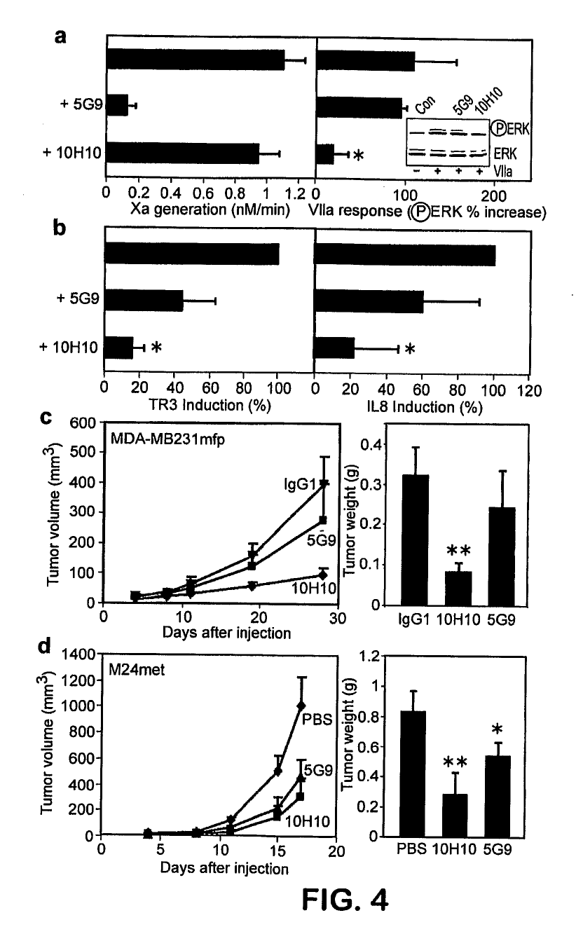

Specific antibodies to coagulant (MAb-5G9) and signaling (MAb-10H10) TF provided an opportunity to address the roles of these respective activities in tumour progression. Specific targeting of tumour cell TF was achieved in the xenograft MDA-MB231 breast cancer model. The antibodies showed the expected specificities to suppress coagulation in the case of MAb-5G9, whereas MAb-10H10 inhibited signaling, including the induction of proangiogenic interleukin 8 and TR3, a typical early response gene downstream of PAR signaling (FIGS. 4a,b). Tumour cells implanted with MAb-10H10, but not MAb-5G9, showed significantly reduced final tumour sizes and tumour weights relative to isotype matched control IgG1 (FIG. 4c). Antibody-treated cells grew indistinguishable from controls in tissue culture, consistent with previous results that TF expression has no effect on in vitro proliferation. Yu et al., Blood 105: 1734-1741, 2005; Zhang et al., J. Clin. Invest. 94: 1320-1327, 1994. MAb-5G9 slightly reduced tumour volumes, consistent with MAb-5G9's partial inhibitory effect on TF-VIIa signaling in vitro.

TF supports the early arrest phase of experimental melanoma metastasis through thrombin pathways, because MAb-5G9, but not MAb-10H10 suppressed melanoma M24met metastasis. Mueller et al., Proc. Natl. Acad. Sci. USA 89: 11832-11836, 1992. Because the regiment of antibody administration in previous experiments were insufficient to evaluate tumour growth of metastases, we revisited the role of TF signaling in tumour expansion of this melanoma model. MAb-5G9 retarded melanoma primary tumour growth, but MAb 10H10 was more potent to reduce both final tumour volumes and tumour weights (FIG. 4d). In contrast to coagulation-driven haematogenous metastasis, targeting TF-VIIa signaling thus efficiently suppressed primary growth of two independent tumour models in vivo.

FIG. 4 shows TF-VIIa signaling promotes tumour growth. a, b Signaling of TF-VIIa is blocked by MAb 10H10, but not MAb 5G9 in MDA-MB231 breast cancer cells (* p<0.01, t-test, mean±sd (n>4). Inset: representative experiment. c MDA-MB231 primary tumour growth in the presence of 1 mg of control IgG1 (TIB115), MAb-5G9 or MAb-10H10 coninjected to achieve high local antibody concentrations. Tumour weights at sacrifice (n=6, 2-sided ANOVA, Kruskall Wallis ** p<0.001). d Tumour growth of M24met melanoma cells implanted without or with 1 mg MAb-5G9 or 10H10 (n=8, 2-sided ANOVA, Kruskall Wallis **p<0.001, p<0.01).

These experiments identify the molecular mechanism by which TF is switched from an initiating cofactor of the coagulation cascade to a non-coagulant signaling co-receptor that drives pathology. Because signaling TF-VIIa inefficiently binds substrate, it escapes rapid feedback inhibition by the Xa-dependent TF pathway inhibitor (TFPI). Huang et al., Blood 90: 944-951, 1997. Thereby, pathophysiological upstream TF-VIIa signaling proceeds without typical inhibitory circuits that follow coagulation activation. Targeting signaling TF suppresses tumour growth, providing direct evidence that TF-VIIa mediated PAR2 activation is a central signaling pathway that drives pathologies independent of thrombin signaling in vivo. The example of TF shows that disulfide exchange pathways have the versatility to switch a single receptor between two distinct biological functions and that such a regulatory switch may be exploited for potential therapeutic benefit. This study should encourage similar targeting of other pathophysiologically relevant cell surface receptors.

### Example 5

### Epitope Assignment for Antibody Specific to Signaling Tissue Factor

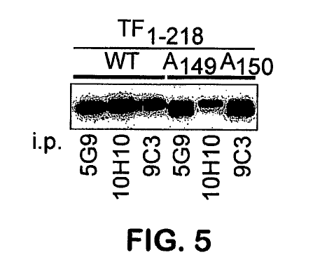

FIG. 5 shows an epitope assignment for MAb-10H10, a monoclonal antibody that binds specifically to signaling tissue factor. The figure show immunoprecipitation of wild-type or mutated soluble TF1-218 with the indicated antibodies followed by detection by Western blotting. MAb-10H10 is inefficient in immunoprecipitating TF that is mutated at residues 149 and 150 (A149, A150), indicating epitope localization in the carboxyl-terminal domain of TF close to or directly involving these residue side chains.

### Example 6

### Nitric Oxide (NO) Dependent Suppression of TF Coagulant Activity by PDI

As noted above, the mutational analysis showed that breaking the Cys¹⁸⁶-Cys²⁰⁹ disulfide renders TF coagulation inactive, but MPB labeling showed no prominent free thiols of TF on high Ca²⁺ cells. Cell surface PDI catalyzes trans-nitrosylation and de-nitrosylation reactions and PDI can be S-nitrosylated at vicinal thiols. 50-100 μM Hg²⁺ was required to enhance TF coagulant activity, which are typical concentrations to release nitric oxide (NO) from PDI (Sliskovic et al., J. Biol. Chem. 280, 8733-8741, 2005), raising the possibility that the activating effect of Hg²⁺ involved denitrosylation of PDI. To assess this possibility, the biotin switch method was used to detect S-nitrosylation. After blocking cellular free thiols with N-ethylmaleimide (NEM), cells were MPB-labeled in the presence or absence of ascorbic acid to release NO. Similarly, after blockade of free thiols with 1 mM iodacetamide, MPB-labeling of TF immunoprecipitates in the presence of ascorbic acid was measured.

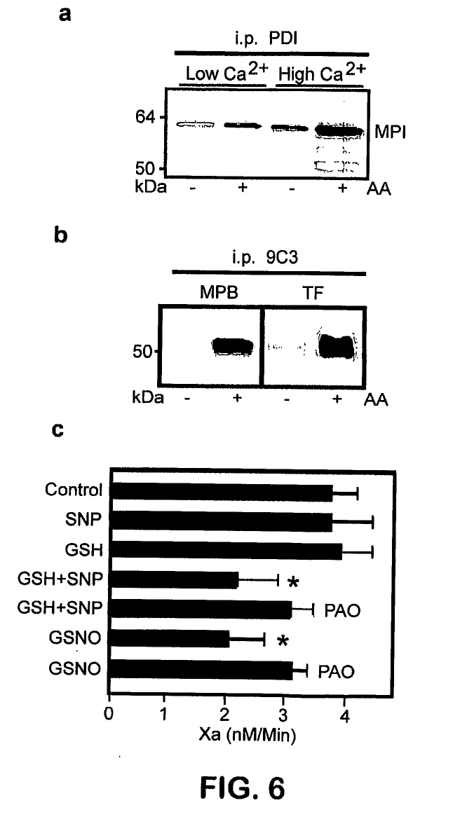

As shown in FIG. 6, results from these studies indicate that PDI suppresses TF coagulant activity in a nitric oxide-dependent pathway, linking the regulation of TF thrombogenicity to oxidative stress in the vasculature. FIG. 6a shows that the biotin switch method after thiol blockade with 1 mM N-ethylmaleimide (NEM) detects increased labeling of PDI upon NO release by ascorbic acid (AA) specifically in PDI immunoprecipitates from high Ca²⁺ cells. With specificity for high Ca²⁺ cells, immunoprecipitated PDI showed increased MPB-labeling in the presence of ascorbic acid. MPB-labeled bands at the appropriate molecular weight for TF also became visible in PDI immunoprecipitates. FIG. 6b shows S-nitrosylation of TF detected by the biotin switch method after thiol blockade with 1 mM iodoacetamide prior to MPB-labeling with or without AA. After blockade of free thiols with 1 mM iodacetamide, MPB-labeling of TF immunoprecipitates significantly increased in the presence of ascorbic acid.

It was further tested whether Hg²⁺-induced activation of TF was reversible. After washout of the oxidant, coagulant activity of TF remained high. The results as shown in FIG. 6c demonstrate that Hg²⁺-induced activation of TF coagulant activity is reversible by NO-dependent PDI pathways. In this experiments cells washed after brief 100 μM Hg²⁺ exposure were incubated in HEPES buffer, 1.5 mM Ca²⁺ in the presence of 1 mM sodium nitroprusside (SNP), 1 mM reduced glutathione (GSH), or 1 mM S-nitrosoglutathion (GSNO) with or without 10 μM PAO for 10 minutes prior to Xa generation assay. The data indicate that addition of reduced glutathione (GSH) or the NO donor sodium nitroprusside (SNP) alone had no effect on TF activity, but in combination TF coagulant activity was suppressed (FIG. 6c; * different control (p<0.05, t-test; mean±sd; n=3)). The vicinal thiol blocker PAO prevented inactivation of TF, indicating involvement of PDI to break the TF disulfide. NO reacts with GSH to yield S-nitrosoglutathione and addition of S-nitrosoglutathione was sufficient to suppress TF coagulant activity.

### Example 7

### TF-VIIa Signaling Requires TF.PAR2 Complex Formation

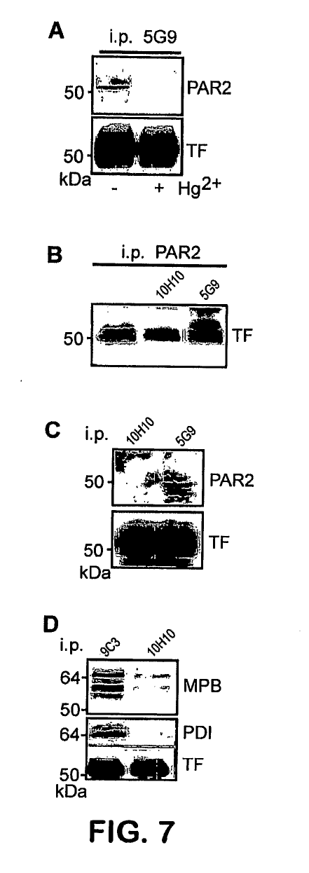

Additional studies as illustrated in FIG. 7 indicate that TF-VIIa signaling requires TF.PAR2 complex formation. FIG. 7a shows that MAb-5G9 immunoprecipitation of PAR2 from high Ca²⁺ cells is abolished by Hg²⁺ pretreatment. FIG. 7b shows that MAb-10H10, but not MAb-5G9, perturbs the TF-PAR2 complex. HaCaT cells were pretreated in serum-free medium for 15 minutes with the indicated antibody and TF was detected in the PAR2 immunoprecipitate. Loading controls were not feasible due to background, because no PAR suitable antibody from a different species was available for Western-blotting. FIG. 7c shows that MAb-10H10 does not immunoprecipitate PAR2 form HaCaT cells. FIG. 7d shows MAb-10H10 does not immunoprecipitate a complex containing PDI. MPB-labeled cells were immunoprecipitated with MAb-9C3 or MAb-10H10 and probed for PDI, TF or thiol-biotinylation with MPB.

As demonstrated in FIG. 7, MAb-5G9 had no effect on TF-VIIa signaling, but showed strong reactivity with coagulant and non-coagulant pools of TF. This antibody immunoprecipitated a complex of TF with PAR2 from HaCaT cells (FIG. 7a). Hg²⁺ treatment to release surface PDI from TF abolished PAR2 complex formation with TF in MAb-5G9 immunoprecipitates. Pretreatment of cells with MAb-5G9 had no effect on the reverse association of TF with PAR2 immunoprecipitates, but the signaling blocking MAb-10H10 association (FIG. 7b). Thus, antibody blockade of TF-VIIa signaling was correlated with reduced TF-PAR2 co-immunoprecipitation. Unlike MAb-5G9, MAb-10H10 did not immunoprecipitate PAR2 (FIG. 7c) or PDI (FIG. 7d), indicating that this antibody prevents the formation of a complex involving TF, PAR2 and PDI. Taken together, these data described in FIG. 7 and FIG. 2 show in a cellular model with physiological levels of TF and PAR2 that PDI acts as a regulatory switch between direct TF-VIIa cell signaling and coagulation activation.

### Example 8

### Materials and Methods

Reagents. Coagulation factors, inhibitors, and antibodies were described previously or from the following suppliers: anti-PDI RL90 (Alexis), SPA-890 (Stressgen), anti-ERP57 (Upstate), N′-(3-maleimidyl propionyl) biocytin (MPB) (Molecular Probes), bacitracin, phenylarsine oxide (Sigma). Riewald and Ruf, Proc. Natl. Acad. Sci. USA 98: 7742-7747, 2001; Ahamed and Ruf, J. Biol. Chem. 279: 23038-23044, 2004; Sevinsky et al., J. Cell Biol. 133: 293-304, 1996; Ruf et al., Biochem. J. 278: 729-733, 1991; Morrissey et al., Thromb. Res. 52:247-61, 1988; Dorfleutner et al., Mol. Biol. Cell 15: 4416-4425, 2004; and Dorfleutner and Ruf, Blood 102: 3998-4005, 2003. Bacitracin was repurified by gel filtration and tested for absence of PDI degradation. Rabbit anti-PAR2 was raised against KLH-coupled peptide TIQGTNRSSKGRSLIGKVDGTSHVTGCG (SEQ ID NO:5). Soluble TF mutants were expressed in E. coli as described in Stone et al., Biochem. J. 310: 605-614, 1995.

In addition to the disclosures in the scientific literatures noted above, the 5G9 and 10H10 antibodies are also described extensively in U.S. Pat. Nos. 5,223,427 and 6,001,978. Hybridomas producing these two antibodies have been deposited pursuant to Budapest Treaty requirements with the ATCC on Mar. 27, 1987 and assigned accession numbers HB9382 and HB9383, respectively.

Cell Culture. HUVECs were maintained and transduced, as described. Ahamed and Ruf, J. Biol. Chem. 279: 23038-23044, 2004. Human HaCaT keratinocyte standard culture was DMEM, 10% FBS, 2 mM glutamine. For low Ca²⁺ culture, cells were split into keratinocyte-SFM (Invitrogen), 10% calcium-depleted FBS for 48 hours and switched by adding 2 mM Ca²⁺ for 24 hours with or without cycloheximide (50 μM). For siRNA knockdown, HaCaT cells were transfected daily at 40% confluence with 100 nM siRNA (Santa Cruz Biotechnology) using 2 μl Lipofectamin 2000 (Gibco).

Functional and signaling assays. For signaling experiments, cell were equilibrated to serum free conditions for 5 hours in M199, 2 mM glutamine, 10 mM HEPES, 1.5 mM Ca²⁺ (HUVECs) or for 24 hours (MDA-MB231) or 10-20 minutes (HaCaT) in DMEM, 2 mM glutamine, 10 mM HEPES. Inhibitors were added 10 minutes prior to stimulation: anti-TF (50 μg/ml), rabbit anti-PAR2 (100 μg/ml), anti-TF (ATAP2, 20 μg/ml, WEDE15, 40 μg/ml), bacitracin (3 mM). Hirudin (200 nM) was added routinely to exclude thrombin signaling. MAP kinase phosphorylation after 10 minutes and gene induction after 90 minutes of agonist stimulation were quantified by Westernblotting or time PCR. Ahamed et al., Blood 105: 2384-2391, 2005.

Cell surface labelling, immunoprecipitation, confocal imaging Cells were washed and labelled in HEPES buffer with 1.5 mM calcium chloride with either 100 μM MPB or 0.5 mg/ml NHS biotin at 4° C. for 20 min. After quenching (1 mM reduced glutathione for MPB, Tris for NHS) immunoprecipitations from 50 mM n-Octyl-β-D-glucopyranoside lysates used MAbs directly coupled to Dynabeads for Westernblotting with goat anti-TF, anti-PDI RL90, or streptavidin-conjugated horseradish peroxidase for biotin detection. Blots were digitized for densitometry using NIH Image Scion. Cells were stained on ice with directly conjugated antibodies for confocal microscopy using a Nikon TE2000-U microscope. Optical sections of each fluorophor were merged using Adobe Photoshop.

Tumour Growth Experiment. 2×10⁶ MDA-MB231mfp cells or 0.5×10⁶ M24met cells were mixed with 1 mg MAb-10H10, MAb-5G9 or isotype matched IgG1 (TIB115) in 100 μl PBS and injected subcutaneously into 6 week-old, female C.B-17 SCID mice (Taconic). Jessani et al., Proc. Natl. Acad. Sci. U.S.A 101: 13756-13761, 2004; Mueller et al., Proc. Natl. Acad. Sci. USA 89: 11832-11836, 1992. Tumour volumes were measured with callipers and at sacrifice tumour weights were determined. ANOVA was used to establish differences between groups and significance levels were determined by non-parametric Kruskal-Wallis test.

When ranges are used herein for physical properties, such as molecular weight, or chemical properties, such as chemical formulae, all combinations and subcombinations of ranges and specific embodiments therein are intended to be included.

All publications, sequences, patents and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

We claim:

1. A method for inhibiting or suppressing tissue factor/factor VIIa (TF/VIIa) signaling in a mammalian subject, the method comprising administering an inhibitor of tissue factor signaling to the mammalian subject, wherein the inhibitor does not interfere with hemostasis in the mammalian subject.

2. The method of claim 1 wherein the subject suffers from an angiogenesis-related disease, a neoplastic disease, or inflammation.

3. The method of claim 1 wherein the inhibitor does not prevent coagulation activation.

4. The method of claim 1 wherein TF/VIIa signaling is dependent on protein disulfide isomerase (PDI).

5. The method of claim 1 wherein TF/VIIa signaling occurs via protease activated receptor 2 (PAR2).

6. The method of claim 1 wherein the inhibitor is an antibody or small chemical entity.

7. The method of claim 6 wherein the inhibitor is an antibody or an antigen-binding molecule having the binding specificity of monoclonal antibody 10H10 produced by the hybridoma with ATCC access number HB9383.

8. The method of claim 6 wherein the inhibitor is monoclonal antibody 10H10 produced by the hybridoma with ATCC access number HB9383.

9. The method of claim 1 wherein the inhibitor inhibits the binding of to tissue factor by monoclonal antibody 10H10 produced by the hybridoma with ATCC access number HB9383.

10. The method of claim 1 wherein the inhibitor does not inhibit the binding to tissue factor by monoclonal antibody 5G9 produced by the hybridoma with ATCC access number HB9382.

11. A method for treating or ameliorating the symptoms of a disease that is dependent upon tissue factor-factor VIIa (TF/VIIa) signaling in a mammalian subject, comprising administering to the subject a therapeutically effective amount of a compound which inhibits TF/VIIa signaling but does not interfere with tissue factor mediated hemostasis activity.

12. The method of claim 11, wherein the disease is a neoplastic disease, an angiogenesis-related disease, or inflammation.

13. The method of claim 11, wherein the disease is a breast tumor or melanoma.

14. The method of claim 11 wherein the hemostasis activity is TF mediated coagulation.

15. The method of claim 11 wherein TF/VIIa signaling is dependent on protein disulfide isomerase.

16. The method of claim 11 wherein TF/VIIa signaling occurs via protease activated receptor 2 (PAR2).

17. The method of claim 11 wherein the compound is an antibody or small chemical entity.

18. The method of claim 17 wherein the compound is an antibody or an antigen-binding molecule having the binding specificity of monoclonal antibody 10H10 produced by the hybridoma with ATCC access number HB9383.

19. The method of claim 17 wherein the compound is monoclonal antibody 10H10 produced by the hybridoma with ATCC access number HB9383.

20. A method for identifying an agent which inhibits TF/VIIa signaling but does not block coagulation, the method comprising measuring in the presence or absence of a test compound the binding between (i) an antibody or an antigen-binding molecule having the binding specificity of monoclonal antibody 10H10 produced by the hybridoma with ATCC access number HB9383 and (ii) a tissue factor polypeptide; and detecting an inhibition of said binding in the presence of the test compound relative to the binding in the absence of the test compound, thereby identifying an agent which inhibits TF/VIIa signaling but does not block coagulation.

21. The method of claim 20, wherein the antibody or an antigen-binding molecule is the monoclonal antibody produced by the hybridoma with ATCC access number HB9383.

22. The method of claim 20, wherein the test compound is small molecule organic compound.

23. The method of claim 20, further comprising detecting an inhibitory effect of the identified agent on tissue factor signaling.

24. The method of claim 20, further comprising detecting no effect of the identified agent on tissue factor-mediated coagulation.

25. The method of claim 20, further comprising (i) contacting, in the presence or absence of the identified agent, a tissue factor polypeptide with the monoclonal antibody 569 produced by the hybridoma with ATCC access number HB9382 and (ii) detecting no inhibition of the identified agent on binding to tissue factor by monoclonal antibody 5G9 produced by the hybridoma with ATCC access number HB9382.
